SEQUENCE LISTING (1 sequence)

SEQ ID NO: 1 - DNA, 2324 bp, Artificial Sequence
  Description of Artificial Sequence Synthetic polynucleotide
ggcgatttcggaggatggagaaatagccccgagtcccgtggaaaatgaggccggcggacttgctgcagctggtgctgctgctcgacctgcccagggacctgggcggaatggggtgttcgtctccaccctgcgagtgccatcaggaggaggacttcagagtcacctgcaaggatattcaacgcatccccagcttaccgcccagtacgcagactctgaagcttattgagactcacctgagaactattccaagtcatgcattttctaatctgcccaatatttccagaatctacgtatctatagatgtgactctgcagcagctggaatcacactccttctacaatttgagtaaagtgactcacatagaaattcggaataccaggaacttaacttacatagaccctgatgccctcaaagagctccccctcctaaagttccttggcattttcaacactggacttaaaatgttccctgacctgaccaaagtttattccactgatatattctttatacttgaaattacagacaacccttacatgacgtcaatccctgtgaatgcttttcagggactatgcaatgaaaccttgacactgaagctgtacaacaatggctttacttcagtccaaggatatgctttcaatgggacaaagctggatgctgtttacctaaacaagaataaatacctgacagttattgacaaagatgcatttggaggagtatacagtggaccaagcttgctggacgtgtctcaaaccagtgtcactgcccttccatccaaaggcctggagcacctgaaggaactgatagcaagaaacacctggactcttaagacactgccctccaaagaaaaattcacgagcctcctggtcgccacgctgacctaccccagccactgctgcgccttcagtaatttgccgaagaaagaacagaatttttcattttccatttttgaaaacttctccaaacaatgcgaaagcacagttagaaaagcagataacgagacgctttattccgccatctttgaggagaatgaactcagtggctgggatgagctcaaaaacccccaggaagagactctacaagcttttgacagccattatgactacaccatatgtggggacagtgaagacatggtgtgtacccccaagtccgatgagttcaacccgtgtgaagacataatgggctacaagttcctgagaattgtggtgtggttcgttagtctgctggctctcctgggcaatgtctttgtcctgcttattctcctcaccagccactacaaactgaacgtcccccgctttctcatgtgcaacctggcctttgcggatttctgcatggggatgtacctgctcctcatcgcctctgtagacctctacactcactctgagtactacaaccatgccatcgactggcagacaggccctgggtgcaacacggctggtttcttcactgtctttgcaagcgagttatcggtgtatacgctgacggtcatcaccctggagcgctggtatgccatcaccttcgccatgcgcctggaccggaagatccgcctcaggcacgcatgtgccatcatggttgggggctgggtttgctgcttccttctcgccctgcttcctttggtgggaataagtagctatgccaaagtcagtatctgcctgcccatggacaccgagacccctcttgctctggcatatattgtttttgttctgacgctcaacatagttgccttcgtcatcgtctgctgctgttatgtgaagatctacatcacagtccgaaatccgcagtacaacccaggggacaaagataccaaaattgccaagaggatggctgtgttgatcttcaccgacttcatatgcatggccccaatctcattctatgctctgtcagcaattctgaacaagcctctcatcactgttagcaactccaaaatcttgctggtactcttctatccacttaactcctgtgccaatccattcctctatgctattttcaccaaggccttccagagggatgtgttcatcctactcagcaagtttggcatctgtaaacgccaggctcaggcataccgggggcagagggttcctccaaagaacagcactgatattcaggttcaaaaggttacccacgacatgaggcagggtctccacaacatggaagatgtctatgaactgattgaaaactcccatctaaccccaaagaagcaaggccaaatctcagaagagtatatgcaaacggttttgtaagttaacactacactactcacaatggtaggggaacttacaaaataatagtttcttgaatatgcattccaatcccatgacacccccaac

(19) United States
(12) Patent Application Publication
(10) Pub. No.: US 2020/0292543 A1
(43) Pub. Date: Sep. 17, 2020

(54) COMPOSITIONS, KITS, AND METHODS FOR DETECTING AUTOANTIBODIES

(71) Applicant: Quidel Corporation, San Diego, CA (US)

(72) Inventors: Richard L. Egan, Oceanside, CA (US); Lynn Yihong Miao, Athens, OH (US); Hannah J. Kim, Athens, OH (US); Jennifer Wendelken, San Diego, CA (US)

(21) Appl. No.: 16/816,009

(22) Filed: Mar. 11, 2020

Related U.S. Application Data
(60) Provisional application No. 62/817,458, filed on Mar. 12, 2019.

(51) Int. Cl. G01N 33/564; G01N 33/50; C12N 15/85; C12N 5/071; G01N 21/76

(52) U.S. Cl. CPC G01N 33/564; G01N 33/5044; C12N 2503/02; C12N 5/0682; G01N 21/76; C12N 15/85

(57) ABSTRACT

Kits, compositions, and methods useful in the diagnosis of thyroid diseases involving autoantibodies are provided.

### CROSS-REFERENCE TO RELATED APPLICATIONS

This application claims the benefit of U.S. Provisional Application No. 62/817,458, filed Mar. 12, 2019, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Feb. 27, 2020, is named 041896-1182_8141_US00_SL.txt and is 3,479 bytes in size.

### TECHNICAL FIELD

The subject matter described herein relates to kits compositions, and methods useful in the diagnosis of thyroid diseases involving autoantibodies.

### BACKGROUND

Thyroid dysfunction affects an estimated 1 to 10 percent of adults in the general population. Many thyroid diseases, including Graves' disease, Hashimoto's thyroiditis, hyperthyroidism, hypothyroidism (including neonatal hypothyroidism), nongoitrous hypothyroidism, euthyroid or hypothyroid autoimmune thyroiditis, and primary myxedema and idiopathic myxedema, involve the action of autoantibodies (thyroid stimulating immunoglobulins (TSIs) and/or thyroid blocking immunoglobulins (TBIs) that recognize and bind to receptors present on the thyroid gland, resulting in undesirable changes in thyroid hormone production.

While diagnostic techniques are available to detect these autoantibodies, many of these techniques are cumbersome, laborious, cannot distinguish between TSI and TBI and/or lack sufficient sensitivity and/or specificity.

### BRIEF SUMMARY

The following aspects and embodiments thereof described and illustrated below are meant to be exemplary and illustrative, not limiting in scope.

The present disclosure provides kits, compositions, and methods for the detection of and/or distinguishing between of thyroid-stimulating antibodies (TSIs) and thyroid-blocking antibodies (TBIs).

In one aspect, provided are kits comprising transgenic cells stably transfected with a first expression vector and a second expression vector, and a reaction buffer with no cell lysing agent, the buffer optionally including a substrate for a reporter. In these provided kits, the first expression vector comprises a nucleotide sequence that encodes a chimeric or wild-type TSH receptor, while the second expression vector comprise a synthetic nucleotide sequence that encodes the reporter, and the synthetic nucleotide sequence (1) is operably linked to a cAMP-inducible promoter inducible and/or (2) further encodes a heterologous cAMP-binding protein, wherein the cAMP-binding protein is fused to the reporter. In these provided kits, expression of the reporter is associated with an intracellular signal that is detected.

In some embodiments, the intracellular signal is detected without lysing the transgenic cells. In other embodiments, the intracellular signal is produced intracellularly and is detected extracellularly. In other embodiments, the reporter is a protein that is continuously expressed to generate a reporter that produces a signal intracellularly upon catalytic reaction with a substrate.

In another aspect, provided are methods for detecting thyroid stimulating and/or thyroid blocking autoantibodies in a sample, comprising: (a) contacting transgenic cells with a sample suspected of comprising thyroid stimulating and/or thyroid blocking autoantibodies, wherein the transgenic cells are stably transfected with a first expression vector and a second expression vector, and (b) detecting an intracellular signal associated with expression of a reporter. In these provided methods, the first expression vector comprises a nucleotide sequence that encodes a chimeric or wild-type TSH receptor, while the second expression vector comprise a synthetic nucleotide sequence that encodes the reporter, and the synthetic nucleotide sequence (1) is operably linked to a cAMP-inducible promoter and/or (2) further encodes a heterologous cAMP-binding protein, wherein the cAMP-binding protein is fused to the reporter.

### BRIEF DESCRIPTION OF THE DRAWINGS

FIG. 1 shows signal-to-background ratios (S/B) for neat vs. diluted samples (normal, low-TSI, and high-TSI) obtained from a presently disclosed in vivo assay for TSIs/TBIs. (see Example 1.)

FIG. 2 shows results from an experiment comparing protocols that include a wash step versus excluding a wash step. FIG. 2 shows signal-to-background ratios for samples (normal, low-TSI, and high-TSI) obtained from a presently disclosed in vivo assay for TSIs/TBIs. (See Example 1.)

FIGS. 3A-3C show results from an experiment comparing protocols that include an overnight cell-seeding step versus a two-hour cell-seeding step. FIG. 3A shows a titration curve and calculated EC₅₀ values using an antibody standard (thyroid-stimulating antibody M22). FIG. 3B shows signal-to-reference ratios for four negative (NS1, NS2, NS3, and NS4) and four positive (PS1, PS2, PS3, and PS4) samples. FIG. 3C shows responses for each positive sample, calculated as fold response over average values of negative samples. (See Example 1.)

FIGS. 4A-4C show results from an experiment comparing protocols that include a 2-hour cell-seeding step versus using cells immediately or soon after thawing. FIG. 4A shows a titration curve and calculated EC₅₀ values obtained from M22 standards. FIG. 4B shows signal-to-reference ratios for four negative (NS1, NS2, NS3, and NS4) and four positive (PS1, PS2, PS3, and PS4) samples. FIG. 4C shows responses for each positive sample, calculated as fold response over average values of negative samples. (See Example 1.)

FIG. 5A shows a titration curve and calculated EC50 values obtained from the M22 standards. (See Example 1.)

FIG. 5B shows signal-to-reference ratios for four negative and four positive samples.

FIG. 5C shows responses for each positive sample, calculated as fold response over average values of negative samples. (See Example 1.)

FIG. 6 depicts results from an experiment to assess the specificity of a presently disclosed TSI assay, and shows signal response ratios (SRR) observed using a presently disclosed TSI assay performed on serial dilutions of a thyroid-blocking antibody (K170) or a thyroid-stimulating antibody (M22). (See Example 1.)

FIG. 7 shows real time measurements of results from a rapid TSI assay. A normal serum and three TSI-positive samples were tested by a presently disclosed rapid assay. Results (% SRR) were calculated using the RLU data measured every 10 minutes up to 90 minutes. (See Example 1.)

FIG. 8 depicts results from an experiment to assess whether TSHR blocking antibodies also react with the ChR4 chimeric TSHR receptor. FIG. 8 depicts the percent inhibition of thyroid-blocking antibodies K170, 3H10, and 4C1 observed in a presently disclosed TSI assay. (See Example 2.)

FIG. 9 shows results from experiments comparing protocols with different incubation times (10 minutes, 20 minutes, 30 minutes, 40 minutes, 50 minutes, 60 minutes, 70 minutes, 80 minutes, or 90 minutes). The first three bars for each time point (samples DLS 004, DLS 052, and DLS 034) correspond to signals from normal samples; the last four bars for each time point (samples DLS 079, DLS 060, DLS 016, and DLS 122) correspond to signals from TSI-positive samples. (See Example 2.)

FIG. 10 shows a titration curve and calculated IC₅₀ values obtained from the TSHR blocking antibody K170 on different densities of CHO-ChR4/22F transgenic cells. (See Example 3.)

FIGS. 11A and 11B show results from experiments designed to determine sensitivity of presently disclosed methods. FIGS. 11A and 11B show titration curves and calculated EC₅₀ values for the presently disclosed assay and for the THYRETAIN®TSI assay, respectively. The signals obtained from M22 standards using the presently disclosed methods were about ten times higher than those obtained using the THYRETAIN® TSI assay. The analytical sensitivities of the two assays were similar. (See Example 4.)

FIG. 12 shows results in clinical (human serum) samples using different incubation conditions (1) room temperature for 90 minutes (“RT90”) or (2) 37° C. for 1 hour, and then room temperature for 30 minutes (“37° C.-60/RT30”). For comparison, FIG. 12 also shows results on the same samples using the THYRETAIN® TSI assay. (See Example 5.)

FIG. 13 shows endpoint measurements from assays using either CHO-ChR4/22F transgenic cells or pre-equilibrated CHO-ChR4/22F transgenic cells, and, for comparison, results from the THYRETAIN® TSI assay. (See Example 5.)

FIGS. 14A-14D show kinetic measurements from assays using either CHO-ChR4/22F transgenic cells or pre-equilibrated CHO-ChR4/22F transgenic cells, and for comparison, results from the THYRETAIN® TSI assay, where the kinetic measurements were taken at various time points between 5 and 90 minutes of incubation time. (See Example 5.)

FIGS. 15A-15B depicts % SRR in 130 anti-thyroid peroxidase (TPO) antibody-positive serum samples. In FIG. 15A, which depicts results from the THYRETAIN® TSI assay, the black dotted line indicates the assay cutoff of 140% SRR. In FIG. 15B, which depicts results from the presently disclosed TSI assay, the purple dotted line indicates the preliminary assay cutoff of 31% SRR. (See Example 6.)

FIG. 16 depicts standard curves generated using the World Health Organization (WHO) International Standard for TSI for use with the presently disclosed TSI assay. (See Example 7.)

FIG. 17 shows Table 4 which provides a summary of results of a THYRETAIN® TSI Assay (see Example 5).

FIG. 18 shows Table 5 which provides a summary of results of a THYRETAIN® TSI Assay using CHO-ChR4/22F cells (see Example 5).

FIG. 19 shows Table 6 which provides a summary of results of a THYRETAIN® TSI Assay using pre-equilibrated CHO-ChR4/22F cells (see Example 5).

FIG. 20 provides the nucleotide sequence of a synthetic artificial nucleic acid encoding ChR4 chimeric thyroid stimulating hormone receptor (TSHR), as provided in SEQ ID NO: 1.

### DETAILED DESCRIPTION

The present disclosure provides kits, compositions, and methods for detecting thyroid hormone blocking immunoglobulin (TBI) and/or thyroid stimulating immunoglobulin (TSI). Compared to methods known in the art for detecting TSIs and/or thyroid blocking immunoglobulins (TBIs), presently disclosed methods involves fewer steps and shorter turnaround time, while retaining sensitivity and specificity for TSI and/or TBIs. For example, presently disclosed kits, compositions, and methods allow detection of TSIs and/or TBIs without cell lysis, which not only reduces processing time, but also allows kinetic measurements over time in addition to endpoint measurements.

These features enable, among other things, large-scale use of presently disclosed kits, compositions, and methods. Moreover, the presently disclosed kits, compositions, and methods, are more accessible to a wider range of potential users at various settings.

### I. Definitions

As used herein, the terms “about” and “approximately,” in reference to a numerical value, is used herein to include numbers that fall within a range of 20%, 10%, 5%, or 1% in either direction (greater than or less than) the numerical value unless otherwise stated or otherwise evident from the context (except where such number would exceed 100% of a possible value).

As used herein, the term “polypeptide” generally has its art-recognized meaning of a polymer of at least three amino acids. However, the term also refers to specific functional classes of polypeptides, such as, for example, luciferase polypeptides. For each such class, the present specification may refer to known reference polypeptides having defined sequences. Those of ordinary skill in the art will appreciate, however, that the term “polypeptide” is intended to be sufficiently general as to encompass not only polypeptides having the complete sequence of the referenced polypeptide, but also to encompass polypeptides that represent functional fragments (i.e., fragments retaining at least one activity) of such complete polypeptides. Moreover, those of ordinary skill in the art understand that protein sequences generally tolerate some substitution without destroying activity. Moreover, circular permutations of protein sequences may also retain activity. Thus, any polypeptide that retains activity and shares at least about 30-40% overall sequence identity (including circular permutations), often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Those of ordinary skill in the art can identify other regions of similarity and/or identity by analyzing sequences of various polypeptides referenced herein.

### II. Kits

In one aspect, kits are provided for detecting thyroid-stimulating immunoglobulins (TSIs) and/or thyroid-blocking immunoglobulins (TBIs). The kits generally include (a) transgenic cells stably transfected with a first expression vector and a second expression vector and/or (b) a reaction buffer with no cell lysing agent. Generally, the first expression vector comprises a nucleotide sequence that encodes a chimeric thyroid stimulating hormone (TSH) receptor, and the second expression vector comprises a synthetic nucleotide sequence that encodes a reporter and (i) is operably linked to a cAMP-inducible promoter and/or (ii) further encodes a heterologous cAMP-binding protein, wherein the cAMP-binding protein is fused to the reporter. In some embodiments, provided kits further comprise one more standards or control reagents. Provided kits do not include a cell lysing agent and are not intended to be used with a cell lysing agent, in some embodiments.

When kits are used in accordance with methods as further described herein, transgenic cells express a chimeric TSHR on their cell surfaces. Upon binding to a TSIs and/or TBIs, cAMP levels in the transgenic cells increase, which leads to (1) expression of the reporter and/or (2) binding of cAMP to a fusion protein including the reporter. In some embodiments, binding of cAMP to the fusion protein leads to a conformational change in the reporter, and the conformational change allowed the reporter to be detected or increases the detectability of the reporter.

A. Transgenic Cells

1. First Expression Vector

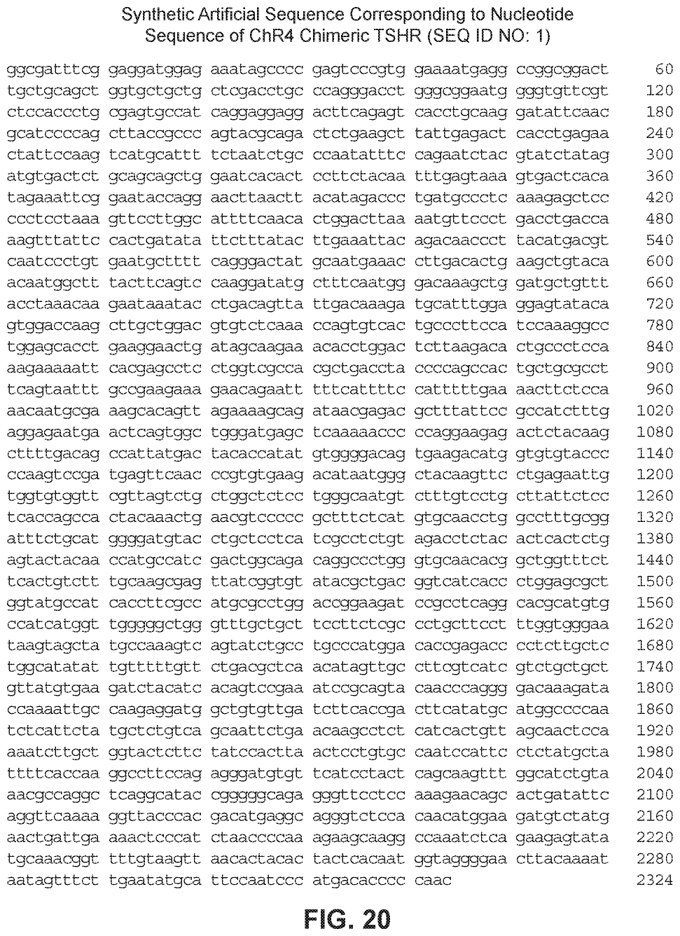

The first expression vector comprises a nucleotide sequence that encodes a chimeric thyroid stimulating hormone (TSH) receptor (chimeric TSHR). Generally, such chimeric TSHRs bind to TSIs, TBIs, or both. In some embodiments, the chimeric TSHR comprises a portion of human TSHR (hTSHR) and a portion of another receptor. For example, the chimeric TSHR may be a chimera of human TSHR (hTSHR) and a luteinizing hormone chorionic gonadotropin receptor (LH-CGR). Non-limiting examples of suitable hTSHR/LH-CGR chimeric receptors include (1) a chimeric receptor having amino acid residues 8-165 substituted by equivalent residues from rat LH-CGR (hereinafter “ChR1”); (2) a chimeric receptor having amino acid residues 90-165 substituted by equivalent residues from rat LH-CGR) (hereinafter “ChR2”), (3) a chimeric receptor having amino acid residues 262 to 335 of hTSHR substituted with equivalent residues from a rat LH/CGR (hereinafter “ChR3”); and (4) a chimeric receptor having amino acid residues 262-368 of the hTSHR substituted by residues 262-334 from rat luteinizing hormone/choriogonadotropin receptor (LH/CGR), hereinafter “ChR4”). (See, e.g., U.S. Pat. No. 9,739,775, the entire contents of which are herein incorporated by reference). A non-limiting example of a suitable TSHR is a TSHR whose nucleotide sequence is at least 75%, at least 80%, at least 85%, at least 90%, at least 95%, at least 97.5%, or 100% identical to the nucleotide sequence of SEQ ID NO: 1, and also provided in FIG. 20.

Generally, the chimeric TSHR is operably linked to a promoter. In some embodiments, the promoter is a constitutive promoter.

2. Second Expression Vector

The second expression vector comprises a synthetic nucleotide sequence that encodes a reporter, as further described herein.

Generally, the synthetic nucleotide sequence is operably linked to a promoter. In some embodiments, the synthetic nucleotide sequence is operably linked to a constitutive promoter.

In some embodiments, the synthetic nucleotide sequence is operably linked to a cAMP-inducible promoter; thus, in some embodiments, presence of cAMP induces expression of the reporter.

In some embodiments, the synthetic nucleotide sequence further encodes a heterologous cAMP-binding protein, wherein the cAMP-binding protein is fused to the reporter. In some such embodiments, presence of cAMP induces a conformational change in the reporter by binding to the cAMP-binding protein, and the conformational change corresponds to increased activity of the reporter.

3. Cell Lines

A number of cell lines are suitable for generating stably transfected cells, including cell lines used as components of protein expression systems. In some embodiments, the transgenic cells comprise mammalian cells. Non-limiting examples of suitable mammalian cell lines include adenocarcinomic human alveolar basal epithelial cells (e.g., A549 cells), African monkey kidney cells (e.g., COS and Vero cells), baby hamster kidney (BHK) cells, Chinese hamster ovary (CHO) cells, mouse myeloma cells (e.g., J558L, NSO, and Sp2/0 cells), human bone osteosarcoma cells (e.g., U205), human breast cancer cells (e.g., MCF-7), human cervical cancer cells (e.g., HeLa), human embryonic kidney cells (e.g., HEK293), human fibrosarcoma cells (e.g., HT1080), human liver carcinoma cells (e.g., HepG2), human muscle rhabdomyosarcoma RD cells (“human RD cells”), human retinoblastoma cells (e.g., SO-Rb5 and Y79), mouse embryonic carcinoma cells (e.g., P19), mouse fibroblast cells (e.g., L929 and NIH3T3), mouse neuroblastoma cells (e.g., N2a), and any derivatives of the aforementioned cell lines.

In some embodiments, the mammalian cells comprise Chinese hamster ovary (CHO) cells (including any derivatives thereof) or human RD cells (including any derivatives thereof). Non-limiting examples of CHO cell line derivatives include CHO-K1, CHO pro-3, and DHFR-deficient cell lines such as DUKX-X11 and DG44.

In some embodiments, transgenic cells further comprise a substrate for the reporter.

B. Reporters and Substrates

In some embodiments, the reporter's presence can be detected without lysing the transgenic cells. For example, the reporter itself can comprise a detectable moiety such as a fluorescent label. Alternatively or additionally, the reporter can bind to or act on a substrate, and the reporter's binding to or acting on a substrate is detectable intracellularly. For example, the reporter can comprise an enzyme whose action on a substrate is detectable intracellularly.

In some embodiments, the reporter is a light-emitting reporter, e.g., a chemiluminescent or bioluminescent reporter. For example, the reporter may comprise an enzyme whose action on its substrate emits light.

For example, the reporter may comprise a luciferase polypeptide, such as, but not limited to, firefly luciferase (e.g., Photinus pyralis luciferase), Renilla luciferase (e.g., Renilla reform is luciferase), Gaussia luciferase (e.g., Gaussia princeps luciferase), Oplophorus luciferase (e.g., Oplophorus gracilirostris luciferase), or a variant or combination thereof. In some embodiments, the reporter is a modified luciferase. Examples of modified luciferases include, but are not limited to, those described in U.S. Pat. Nos. 5,670,356; 7,729,118; and 8,008,006, the entire contents of each of which are herein incorporated by reference. In some embodiments, the modified luciferase is a circularly permutated luciferase.

In some embodiments, a conformational change in the luciferase polypeptide is associated with increased luciferase activity.

In some embodiments, provided kits further comprise a substrate for the reporter. The substrate may be provided as a separate reagent. Alternatively or additionally, the substrate may be provided as a part of another reagent. For example, as mentioned herein, transgenic cells may comprise the substrate.

Any substrate appropriate for the reporter may be used. In some embodiments, the substrate can diffuse through the cell membrane and into the cytoplasm. In some embodiments, the substrate is actively transported through the cell membrane and into the cytoplasm.

For example, when the reporter comprises a luciferase polypeptide, the substrate may be any corresponding luciferin. A luciferin is a “corresponding luciferin” when the luciferase polypeptide can oxidize the luciferin to generate an excited molecule (e.g., an oxyluciferin), which then emits light when it relaxes to a ground state.

For example, D-luciferin or a derivative thereof can be a substrate for a variety of luciferase polypeptides, including firefly luciferases. As another example, coelenterazine or a derivative thereof can be a substrate for a variety of luciferase polypeptides, including Renilla luciferase, Gaussia luciferase, and Oplophorus luciferase.

C. Reaction Buffers

Reaction buffers lack a cell lysing agent and generally comprise a mixture of salts. For example, reaction buffers may comprise a salt selected from the group consisting of: CaCl₂, KCl, KH₂PO₄, MgSO₄, Na₂HPO₄, NaHCO₃, NaCl, HEPES (4-(2-Hydroxyethyl) piperazine-1-ethanesulfonic acid), or any combination thereof. In some embodiments, reaction buffers comprise CaCl₂, KCl, KH₂PO₄, MgSO₄, Na₂HPO₄, and HEPES. In some embodiments, reaction buffers comprise CaCl₂, KCl, KH₂PO₄, MgSO₄, Na₂HPO₄, NaHCO₃, and NaCl.

Reaction buffers may also comprise one or more ingredients in addition to the mixture of salts. For example, in some embodiments, reaction buffers further comprise sucrose. In some embodiments, reaction buffers comprise at least 5 g/L, at least 10 g/L, at least 15 g/L, or at least 20 g/L of sucrose. In some embodiments, reaction buffers comprise at most 100 g/L, at most 90 g/L, at most 80 g/L, at most 70 g/L, at most 60 g/L, at most 50 g/L, at most 40 g/L of sucrose, at most 30 g/L, or at most 20 g/L of sucrose. In some embodiments, reaction buffers comprise between 5 g/L and 100 g/L, between 10 g/L and 90 g/L, between 10 g/L and 80 g/L, between 10 g/L and 70 g/L, between 10 g/L and 60 g/L, between 10 g/L and 50 g/L, between 10 g/L and 40 g/L, between 10 g/L, or between 10 g/L and 30 g/L of sucrose. In some embodiments, reaction buffers comprise about 5 g/L, about 7.5 gL, about 10 g/L, about 12.5 g/L, about 15 g/L, about 17.5 g/L, about 20 g/L, about 22.5 g/L, about 25 g/L, about 27.5 g/L, about 30 g/L, about 32.5 g/L, about 35 g/L, about 37.5 g/L, about 40 g/L, about 42.5 g/L, about 45 g/L, about 47.5 g/L, about 50 g/L, about 55 g/L, about 60 g/L, about 65 g/L, about 70 g/L, or about 75 g/L of sucrose.

In some embodiments, reaction buffers comprise at least 10 mM, at least 20 mM, at least 30 mM of sucrose, at least 40 mM, at least 50 mM, at least 75 mM, or at least 100 mM of sucrose. In some embodiments, reaction buffers comprise at most 300 mM, at most 275 mM, at most 250 mM, at most 225 mM, at most 200 mM, at most 175 mM, at most 150 mM, at least 125 mM, at most 100 mM, at most 90 mM, at most 80 mM, at most 70 mM, at most 60 mM, or at most 50 mM of sucrose. In some embodiments, reaction buffers comprise between 10 mM and 300 mM, between 20 mM and 200, between 30 mM and 150 mM of sucrose, between 30 mM and 125 mM, or between 30 mM and 100 mM of sucrose. In some embodiments, reaction buffers comprise about 10 mM, about 15 mM, about 20 mM, about 25 mM, about 30 mM, about 35 mM, about 40 mM, about 45 mM, about 50 mM, about 60 mM, about 70 mM, about 80 mM, about 90 mM, about 100 mM, about 120 mM, about 140 mM, about 160 mM, about 180 mM, about 200 mM, or about 220 mM of sucrose.

In some embodiments, reaction buffers further comprise polyethylene glycol (PEG), for example a PEG having a molecular weight of between 100 and 20,000 (e.g., PEG-100, PEG-200, PEG-300, Peg-400, PEG-600, PEG-1000, PEG-1500, PEG-2000, PEG-2050, PEG-3000, PEG-3350, PEG-4000, PEG-4600, PEG-6000, PEG-8000, PEG-10,000, PEG-12,000, PEG-20,000 or mixture thereof). In some embodiments, reaction buffers comprise at least 0.5% PEG, at least 1% PEG, at least 2% PEG, at least 3% PEG, at least 4% PEG, at least 5% PEG, at least 6% PEG, at least 7% PEG, or at least 8% PEG. In some embodiments, reaction buffers comprise at most 12% PEG, at most 11% PEG, at most 10% PEG, at most 9% PEG, at most 8% PEG, at most 7% PEG, at most 6% PEG, or at most 5% PEG. In some embodiments, reaction buffers comprise between 0.5% and 12% PEG, between 1% and 10% PEG, or between 2% and 6% PEG. In some embodiments, reaction buffers comprise about 0.5%, about 1%, about 2%, about 3%, about 4%, about 5%, about 6%, about 7%, or about 8% PEG.

In some embodiments, reaction buffers comprise albumin, e.g., bovine serum albumin. In some embodiments, reaction buffers comprise at least 0.5%, at least 1%, at least 2%, at least 3%, at least 4%, or at least 5% albumin. In some embodiments, reaction buffers comprise at most 12%, at most 10%, at most 8%, at most 6%, at most 4%, or at most 2% albumin.

In some embodiments, reaction buffers comprise salts, PEG, sucrose, and albumin. In some embodiments, reaction buffers comprise salts, PEG, and sucrose, but no albumin. In some embodiments, reaction buffers comprise salts and PEG, but no sucrose or albumin.

Non-limiting examples of reaction buffer formulations include formulations disclosed in U.S. Pat. No. 9,739,775 (therein referred to as “Stimulation Medium”), the entire contents of which are herein incorporated by reference.

In some embodiments, the reaction buffer comprises a reagent that inhibits the binding of TSH to the wild type or chimeric TSHR receptor thereby preventing a false signal due to the presence of normal physiological or higher levels of TSH in, e.g., a test blood sample. The TSH inhibitor may be an antibody that specifically binds TSH, or other agent that blocks the binding of TSH to the wild type or chimeric TSHR without binding to the TSHR, thus allowing the assay to proceed without interference from either TSH or the TSH blocking reagent.

D. Controls and Standards

In some embodiments, provided kits further comprise one or more control thyroid stimulating agents and/or one or more control thyroid blocking agents. As used herein, the term control thyroid-stimulating agent refers to an agent that is known to stimulate TSHRs expressed on a mammalian cell. Binding of a control thyroid-stimulating agent to a TSHR induces intracellular signaling events that include cAMP upregulation. Examples of suitable control thyroid-stimulating agents include, but are not limited to, thyroid-stimulating antibodies (e.g., M22 anti-TSHR mAb and NIBSC 08/204 (the WHO International Standard for thyroid-stimulating antibodies) and thyroid-stimulating hormones (TSH) (e.g., bovine TSH). As used herein, the term control thyroid-blocking agent refers to an agent that is known to block the action of TSHRs expressed on a mammalian cell, e.g., by preventing TSH binding to the TSHR, or by binding to the TSHR and thereby inhibiting the binding of a stimulating agents such as a TSI specific antibody. Examples of suitable control thyroid-stimulating agents include, but are not limited to, thyroid-blocking antibodies such as 3H10 and K170.

In some embodiments, provided kits comprise one or more control samples, e.g., control negative samples lacking TSIs or TBIs, control negative samples comprising control thyroid-blocking agents, and/or control positive samples comprising TSIs, TBIs, or control thyroid-stimulating agents. Non-limiting examples of suitable control negative samples include serum samples, clinical samples (e.g., human serum samples) known to be negative for TSIs and TBIs, artificially made compositions lacking TSIs and TBIs, clinical samples known to comprise control thyroid-blocking agents, or artificially made samples comprising control thyroid-blocking agents. Non-limiting examples of suitable control positive samples include serum samples spiked with TSIs, TBIs, or control thyroid-stimulating agents; clinical samples (e.g., human serum samples) known to be positive for TSIs and/or TBIs, or artificially made compositions spiked with TSIs, TBIs, or control thyroid-stimulating agents.

In some embodiments, provided kits comprise a quantitation standard or set of standards, e.g., for quantitating amount of TSIs and/or TBIs in a sample. The standard may be characterized by a known quantity or concentration of an agent, e.g., control thyroid-stimulating agent and/or a control thyroid-blocking agent. The kit may include instructions for diluting the standard to make set of standards, or the kit may comprise set of standards, each having a different known quantity or concentration of the agent.

When used in accordance with provided methods, in some embodiments, at least one control samples or standard is processed in parallel with other samples.

### III. Assay Methods

In one aspect, methods are provided for detecting thyroid-stimulating immunoglobulins (TSIs) and/or thyroid-blocking immunoglobulins (TBIs). Provided methods generally comprise steps of (a) contacting transgenic cells (as described herein) with a sample suspected of comprising thyroid stimulating and/or thyroid blocking autoantibodies and (b) detecting an intracellular signal associated with expression of the reporter.

In some embodiments, the step of contacting comprises contacting in a buffer that comprises a substrate for the reporter. The buffer generally excludes a cell lysing agent and can be, e.g., any reaction buffer as described above.

In some embodiments, provided methods further comprise a step of exposing the transgenic cells to the substrate for the reporter before the step of contacting.

The sample may be obtained from any subject, as described further herein. In some embodiments, the sample comprises serum. In some embodiments, the sample is an undiluted sample.

In some embodiments, the step of detecting is performed no more than (about or less than) 240 minutes, no more than 180 minutes, no more than 90 minutes, no more than 60 minutes, no more than 30 minutes, no more than 15 minutes, or no more than 5 minutes after the step of contacting. For example, in some embodiments, the step of detecting is performed less 240 minutes, less than 180 minutes, less than 90 minutes, or less than 60 minutes after the step of contacting. In some embodiments, the step of detecting is performed between about 5 minutes and about 60 minutes after the step of contacting, e.g., about 10 minutes, about 15 minutes, 20 minutes, about 25 minutes, about 30 minutes, about 35 minutes, about 40 minutes, about 45 minutes, about 50 minutes, about 55 minutes, or about 60 minutes.

In some embodiments, the step is of detecting is performed after the step of contacting without adding or removing the transgenic cells or the sample. Thus, in some embodiments, the intracellular signal is detected from a composition comprising the transgenic cells and the sample. In some embodiments, at least a portion (e.g., at least 50%, at least 55%, at least 60%, at least 65%, at least 70%, at least 75%, at least 80%, at least 85%, at least 90%, at least 95%, at least 98%,) of the transgenic cells in the composition are intact (i.e., not lysed) at the time of detecting.

In some embodiments, the step is of detecting is performed after the step of contacting without adding or removing the substrate. In some embodiments, the step is of detecting is performed after the step of contacting without adding or removing any of the substrate, the transgenic cells, or the sample.

In some embodiments, the step of contacting is performed at room temperature (e.g., entirely at room temperature). In some embodiments, the method is performed entirely at room temperature—that is, all steps in the method are performed at room temperature.

In some embodiments, provided methods further comprise thawing the transgenic cells before the step of contacting. In some embodiments, the step of contacting is performed less than 120 minutes, less than 90 minutes, less than 60 minutes, or less than 30 minutes after said thawing.

In some embodiments, provided methods are performed using multi-well plates, e.g., 96-well plates, 384-well plates, etc., for example, black, white, white plates with a clear bottom or clear plates. In some embodiments, provided methods are performed using white plates. In some embodiments, provided methods are performed on uncoated or untreated plates.

A. Detecting TSIs

In some embodiments, provided methods are used to detect TSIs. In these embodiments, the chimeric TSHR binds to TSIs and may or may not also bind to TBIs, and the sample is suspected of comprising TSIs and may or may not also be suspected of comprising TBIs.

When provided methods are used to detect TSIs, signals from samples suspected of comprising TSIs are generally compared against a control baseline value. Signals greater than the control baseline value may indicate presence of TSIs.

The control baseline value can be provided (e.g., in instructions included in a provided kit), or it may be obtained from a control well that contains the transgenic cells and the substrate, but does not contain TSIs. For example, the control well include (1) a control sample that does not comprise TSIs and is intended to be processed in parallel with samples suspected of comprising TSIs; and/or (2) a composition that does not need to be processed in parallel with samples suspected of comprising TSIs. Alternatively or additionally, the control well may lack a sample but is otherwise processed in parallel with samples suspected of comprising TSIs.

B. Detecting TBIs

In some embodiments, provided methods are used to detect TBIs. In these embodiments, the chimeric TSHR binds to TBIs and may or may not also bind to TSIs, and the sample is suspected of comprising TBIs and may or may not also be suspected of comprising TSIs.

Generally, when provided methods are used to detect TBIs, the step of contacting further comprises contacting the transgenic cells with a control thyroid-stimulating agent (as further described herein) that binds to the chimeric TSHR that encoded by the first expression vector. In some embodiments, the transgenic cells are contacted with the control thyroid-stimulating agent before the transgenic cells are contacted with the sample.

Generally, when performing an assay to detect TBIs, signals from samples suspected of comprising TBIs are compared against a control baseline value. When the signal associated with a sample is reduced compared to the control baseline value, the sample may comprise TBIs.

The control baseline value can be provided (e.g., in instructions included in a provided kit), or it may be obtained from a control well that contains the thyroid-stimulating agent, the transgenic cells, and the substrate, but does not contain TBIs. For example, the control well include (1) a control sample that does not comprise TBIs and is intended to be processed in parallel with samples suspected of comprising TBIs; and/or (2) a composition that does not need to be processed in parallel with samples suspected of comprising TBIs. Alternatively or additionally, the control well may lack a sample but be otherwise processed in parallel with samples suspected of comprising TBIs.

### IV. Applications

Kits, compositions, and methods provided herein may be used to detect thyroid-stimulating immunoglobulins (TSIs) and/or thyroid blocking antibodies (TBIs). This detection may be useful in the diagnosis of one or more diseases associated with the presence of autoantibodies against TSHR, e.g., TSIs and/or TBIs.

In some embodiments, samples are obtained from subjects suspected of having, or at risk of developing, an autoimmune thyroid disease. In some embodiments, the autoimmune thyroid disease is associated with the presence of TSIs. In some embodiments, the autoimmune thyroid disease is associated with the presence of TBIs. In some embodiments, the autoimmune thyroid disease is associated with the presence of both TSIs and TBI. Some subjects may have more than one disorder, or their disorders may also change over time, such that the profile of any autoantibodies in their system changes over time, e.g., switches from predominantly one type of autoantibody to another type (e.g., TSI to TBI or vice versa.)

For example, hyperthyroidism is often associated with production of TSIs. A non-limiting example of a disease characterized by hyperthyroidism is Graves' disease.

Some hypothyroid disorders are associated with TBIs. Examples of hypothyroid disorders, include, but are not limited to, Hashimoto's disease, neonatal hypothyroidism, nongoitrous hypothyroidism, primary myxedema, and idiopathic myxedema.

In some embodiments, subjects from whom samples are obtained are mammals. In some embodiments, the subjects are humans.

In some embodiments, samples are blood or serum samples.

### V. RELATED KITS and METHODS

One of skill in the art will recognize that the present disclosure provides sufficient guidance to create still other products for the detection and screening of other biological molecules, in addition to TSHR autoantibodies that modify of TSH signaling activity. For example, in view of the teaching herein, one of skill can construct a cell-based reporter assay system for detecting stimulatory or blocking antibodies, or other stimulatory or blocking agents, that have an effect on intracellular signaling that exert their effects through G-protein coupled cell-surface receptors (GPCR), leading to changes in intracellular cAMP levels.

GPCRs are a large family of integral membrane proteins that respond to a variety of extracellular stimuli. Each GPCR binds to and is activated by a specific ligand stimulus that ranges in size from small molecule catecholamines, lipids, or neurotransmitters to large protein hormones. When a GPCR, for example the TSH receptor, is activated by its extracellular ligand TSH, a conformational change is induced in the receptor that is transmitted to an attached intracellular heterotrimeric G protein complex. In a cAMP-dependent pathway, the activated protein Gₛ alpha subunit binds to and activates an enzyme called adenylyl cyclase, which, in turn, catalyzes the conversion of ATP into cyclic adenosine monophosphate (cAMP).

Increases or decreases in concentration of the second messenger cAMP can be detected, for example, by a cAMP-inducible promoter reporter construct within a host transgenic cell that also expresses the relevant GPCR. Alternatively, increases or decreases in concentration of cAMP can be detected by use of a modified/heterologous cAMP-binding protein, where the cAMP binding protein is fused to a reporter moiety that can be detected and/or quantitated.

It is known that a wide range of peptide and polypeptide moieties can be appended to either the N- or C-terminus of GPCR molecules without disrupting substantially the signal transduction activity of the receptor. That observation permits the construction of other detection systems in addition to the TSHR system described in the present disclosure.

In one aspect, it is possible to use the cell-based detection system described herein, with a modified/chimeric GPCR to trigger a signaling cascade (such as a cAMP signal) when the GPRC is modified to include a fusion protein with one or more Mycobacterium tuberculosis (TB) antigens. This system can then detect antibodies that may be present, for example, in the blood of a subject, where the anti-TB antibodies can bind to the TB antigen portion of the chimeric GPCR fusion receptor on the surface of a reporter cell, where the anti-TB antibody binding will result in activation or suppression the GPCR signaling portion of the fusion protein, triggering an increase or decrease in cAMP production.

The assessment of an increase or decrease of cAMP production in response to anti-TB antibodies that bind to the TB-antigen on the chimeric GPCR molecule can be detected, for example, by using the cAMP sensitive promoter reporter construct, or alternatively, a cyclic AMP binding protein fused to a suitable reporter moiety.

In still other embodiments, using this same principle, it is possible to use modified versions of the systems described herein that will enable transgenic detector cells to detect specific T-cells that have been activated by TB antigens that trigger T-cell specific responses. This approach will work for any antigen that initiates a cell-mediated immune response.

Interferon-γ (IFN-γ or gamma) release assays (IGRA) are also find use when used with the methods and kits as described herein, for example, IGRAs for the diagnosis of both latent (LTBI) and active tuberculosis (TB). The IGRA relies on the fact that T-lymphocytes will release IFN-γ when exposed to specific antigens, which is quantitated by an ELISA-type assay. Various commercial IGRAs are available for the diagnosis of TB infection. For example, the QIAGEN® QuantiFERON®-TB Gold assay is a whole blood test to quantitate the amount of IFN-γ that is produced in response to mixtures of two synthetic peptides corresponding to Mycobacterium tuberculosis antigens. In addition, the Oxford Immunotec Ltd. T-SPOT.TB™ IGRA is also available, which counts the number of antimycobacterial effector T cells that produce interferon-gamma in a blood sample in response to exposure to Mycobacterium tuberculosis-specific antigens.

### VI. Identification of Inhibitory Anti-TSHR Autoantibodies (TBI)

Thyroid blocking immunoglobulins (TBI) are autoantibodies that bind to the thyroid stimulating hormone receptor (TSHR) and inhibit the action of thyroid stimulating hormone (TSH). The presence of TBI will lead to Hashimoto's disease, but TBI is not the only cause of Hashimoto's disease.

The ability to distinguish between thyroid stimulating immunoglobulin (TSI) and thyroid blocking immunoglobulin (TBI) requires a biological test system rather than a simple immunoassay, as both TSI and TBI autoantibodies bind to TSHR, therefor making this type serology detection system ineffective as distinguishing inhibitory from stimulatory biological effects. A biological test system that tests for the biological effect of the autoantibody on the TSHR is required.

The present disclosure provides a modification of the cell-based biological assay protocols described herein, where the modified protocols distinguishing specifically TBI autoantibody, in contrast to TSI autoantibody. These protocols include the following modifications:

(1) At the time of the cell-based assay, a controlled amount of a TSI-specific monoclonal antibody (MAb) is added to the test well containing a small aliquot of the patient serum.

(2) In the absence of TBI autoantibody in the patient test serum, there will be stimulation/activation of the cell-based biological test system and detection of the reporter, leading to a predetermined reporter signal range that is designated as a negative signal.

(3) In the presence of TBI autoantibody in the patient test serum, the assay will show a 30% or greater loss of reporter signal (inhibition of signal). This reduction in reporter activity is indicative of the presence of TBI autoantibody in the subject's serum.

The 30% reduction in reporter signal compared to a predetermined “TBI negative signal” is only one embodiment, as any statistically significant reduction in reporter activity is also contemplated as a positive test for the presence of TBI autoantibodies in the patient sera. For example, other quantitative thresholds indicative of the presence of TBI autoantibodies can include any reduction in reporter activity of at least 10%, at least 20%, at least 30%, at least 40%, at least 50%, at least 60%, at least 70%, or at least 80%.

### EXAMPLES

### Example 1

### Assay for Detecting Thyroid-Stimulating Immunoglobulins (TSIs)

This Example describes development of an improved method to detect thyroid-stimulating immunoglobulins (TSIs) in a sample. In the improved method disclosed herein, samples are incubated with doubly transgenic cells expressing (1) a chimeric thyroid stimulating hormone receptor (TSHR) receptor on its cell surface, and (2) a modified luciferase fused to a cAMP binding-protein. Binding of the chimeric TSHR receptor, such as to a thyroid-stimulating immunoglobulin in a sample, leads to cAMP signaling within the transgenic cells. cAMP binds to the cAMP binding-protein, which causes a conformational change in the modified luciferase that enhances the modified luciferase's activity. When the modified luciferase cleaves its substrate, a light signal is generated. Signals are then read from the incubation mixture without lysing the cells.

Thus, this method allows, among other things, kinetic measurements over time (e.g., a homogeneous real-time assay). Moreover, this method is compatible with automation and has a much shorter turn-around time than other TSI and/or thyroid-blocking immunoglobulins (TBI) detection assays. For example, assays performed in accordance with this method may allow results to be read in 90 minutes or less from initiation of a protocol.

### A. Materials and Methods

Chinese Hamster Ovary (CHO) cells were transiently transfected with two plasmids: a first plasmid encoding a GLOSENSOR™ (available from Promega) luciferase and a cAMP receptor (“p22F”), and second plasmid encoding a thyroid stimulating hormone receptor (TSHR). One set of CHO cells was transfected with a plasmid encoding a wild-type TSHR (pTSHR-WT); another set of CHO cells was transfected with a plasmid encoding ChR4 (“pChR4”), a chimeric TSHR comprising human TSHR sequences and rat luteinizing hormone receptor (LHR) sequences. ChR4 is described in U.S. Pat. Nos. 8,986,937 and 9,739,775, the contents of each of which are herein incorporated by reference.

Side-by-side assays were performed on both types of transfected cells (cells transfected with the wt TSHR and those transfected with ChR4. The following samples were tested: a sample containing including 2 mIU/mL bTSH, three TSI-positive serum samples, and a normal serum sample as a negative control. The three TSI-positive samples were selected based on results from a THYRETAIN® TSI assay, in which they showed % SRR values of 285%, 376% and 501%. (The THYRETAIN® TSI cutoff is 140% SRR). Reaction buffer with 6% cAMP reagent was used as the assay blank, and results were reported as a signal over blank (S/B) ratio. Cells transfected with p22F/pTSHR-wt exhibited a significantly higher biological response to bTSH stimulation than cells transfected with p22F/pChR4, but both receptors showed similar activity when tested with low TSI-positive samples (data not shown).

With the moderate or high TSI-positive samples, higher S/B ratios were observed with the TSHR-ChR4 transfected cells than with the TSHR-wt transfected cells. Similar results were obtained when this experiment was repeated (data not shown). Based on the transient transfection results, the TSHR-ChR4 receptor was chosen for further development in the present assays.

To generate stably transfected cell lines expressing both the luciferase reporter and the ChR4 receptor, p22F and pChR4 plasmids were linearized by restriction enzyme digestion and co-transfected into CHO K1 cells. After selection, cells were screened in a TSHR stimulation assay using TsAb Mab M22. Six clones were selected for further cloning by limiting dilution based on the level of M22-induced luminescence. A single clone (2C1E3) had the highest signal-to-background ratio and was chosen for assay development. Cells were frozen either in vials or microtiter plate wells (e.g., black or white 96-well plates) for subsequent use in assays.

Genomic integration of the ChR4 receptor was confirmed by polymerase chain reaction (PCR) amplification using primers designed based on the full-length TSHR gene and sequencing of the amplification product. A PCR product was obtained only from the template of TSHR-ChR4 stable cell line with the expected size of 2.1 kb. To confirm the receptor sequence, the PCR product was sequenced and analyzed using Clone Manager software. The PCR product's deduced amino acid sequence was aligned against the predicted TSHR ChR4 protein sequence and TSHR wild type (wt) protein sequence. Sequence alignment results confirmed integration of the TSHR-ChR4 gene in the genome of the cell line. Cells were thawed, mixed with reaction buffer containing substrate, and transferred to or kept in a 96-well microtiter plate and handled as further described below, depending on the experiment. For many of the experiments described in parts B-E, white 96-well plates were used.

TSI-negative (“normal”), low TSI, and high TSI serum samples were tested undiluted (“neat”), diluted 1:2, or diluted 1:4. TSI levels determined by a THYRETAIN® TSI Reporter Bioassay Kit (Quidel Corporation, Athens, Ohio) (“THYRETAIN®” TSI assay) and assigned as “normal,” “low TSI,” “moderate TSI,” or “high TSI” according to Table 1.

TABLE 1
THYRETAIN ® TSI Assay Ranges

| | Level obtained from |
|---|---|
| TSI classification | THYRETAIN ® TSI assay |
| Normal | <140 |
| Low TSI | 140-279 |
| Moderate TSI | 280-420 |
| High TSI | >420 |

### B. Sample Dilution

To test whether a sample dilution step was needed, CHO-ChR4/22F cells were seeded onto plates overnight (^{˜}17-18 hours) and incubated in CHO growth medium, and then medium was removed. One hundred microliters of 6% GLOSENSOR™ substrate in reaction buffer was added to each well. Ten microliters of sample were added to each well in duplicate. The samples tested in the present example were normal serum, low TSI, or high TSI, and each were tested undiluted (“neat”), diluted 1:2, and diluted 1:4.

Plates were incubated for up to 1 hour at 37° C., and then moved to room temperature. Luminometer readings were then taken from each well.

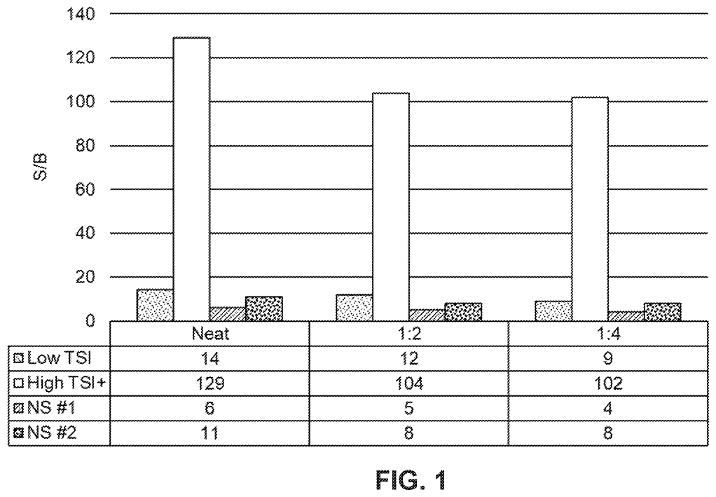

Table 2 and FIG. 1 show results from all samples. Signal-to-background ratios of over 100 were obtained from all high TSI samples (including the “neat” sample), whereas ratios were significantly lower for low TSI and normal serum samples. Moreover, the assay distinguished between samples (e.g., high TSI vs. low TSI or low TSI vs. normal) in undiluted (“neat”) samples at least as well as the assay could in diluted samples.

TABLE 2
Signals from neat and undiluted samples
(S/B = signal-to-baseline ratios)

| Sample | Avg. RLU | S/B Ratio |
|---|---|---|
| Low TSI + Neat | 8073 | 14 |
| Low TSI + 1:2 | 6824 | 12 |
| Low TSI + 1:4 | 4846 | 9 |
| High TSI + Neat | 72385 | 129 |
| High TSI + 1:2 | 58535 | 104 |
| High TSI + 1:4 | 57375 | 102 |
| NS #1 Neat | 3494 | 6 |
| NS #1 1:2 | 2931 | 5 |
| NS #1 1:4 | 2072 | 4 |
| NS #2 Neat | 6217 | 11 |
| NS #2 1:2 | 4544 | 8 |
| NS #2 1:4 | 4294 | 8 |
| Background | 563 | n/a |

Therefore, this assay does not require a sample dilution step.

C. Washing after Cell Seeding

CHO-ChR4/22F cells were seeded as described above in part B of Example 1. Ten microliters of undiluted sample were added to each well in duplicate. For some wells, the seeded cell monolayer was washed with reaction buffer before samples were added. For other wells, no wash step was used before adding the sample. Plates were incubated for up to 1 hour at 37° C., and then moved to room temperature. After 30 minutes at room temperature, luminometer readings were taken from each well.

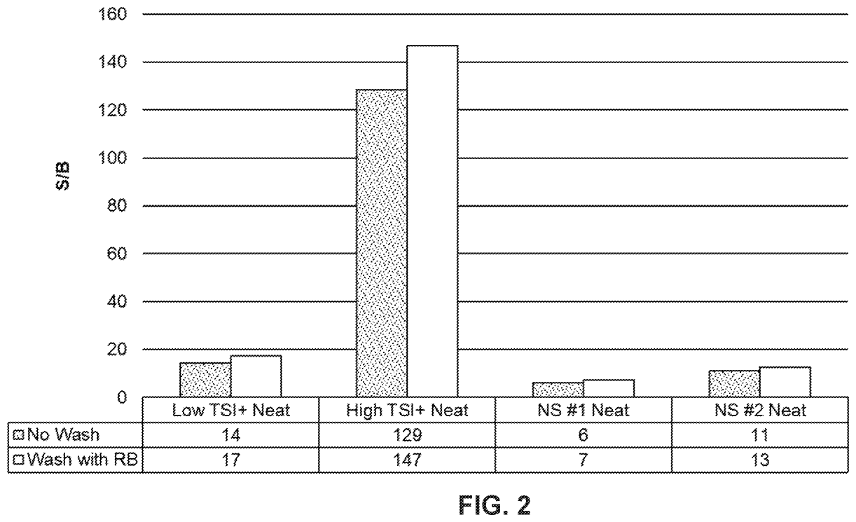

FIG. 2 shows results from this experiment. As FIG. 2 shows, eliminating a wash step did not affect the assay's ability to distinguish between normal, low TSI, and high TSI samples. Therefore, this assay does not require a wash step.

### D. Cell Seeding

To evaluate whether a reduced seeding time would affect assay performance, CHO-ChR4/22F cells were seeded as described in Example 1, Part B either overnight or for 2 hours. Ten microliters of undiluted normal or TSI-positive samples were added to each well in duplicate. Additionally, to generate a titration curve, M22 standards were added to one set of wells. Plates were incubated for up to 1 hour at 37° C., and then moved to room temperature. After 30 minutes at room temperature, luminometer readings were then taken from each well.

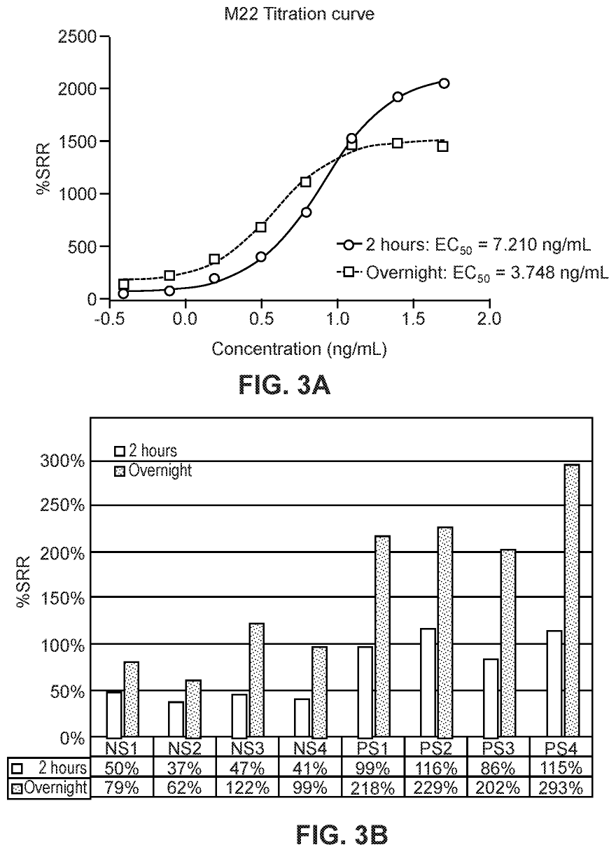

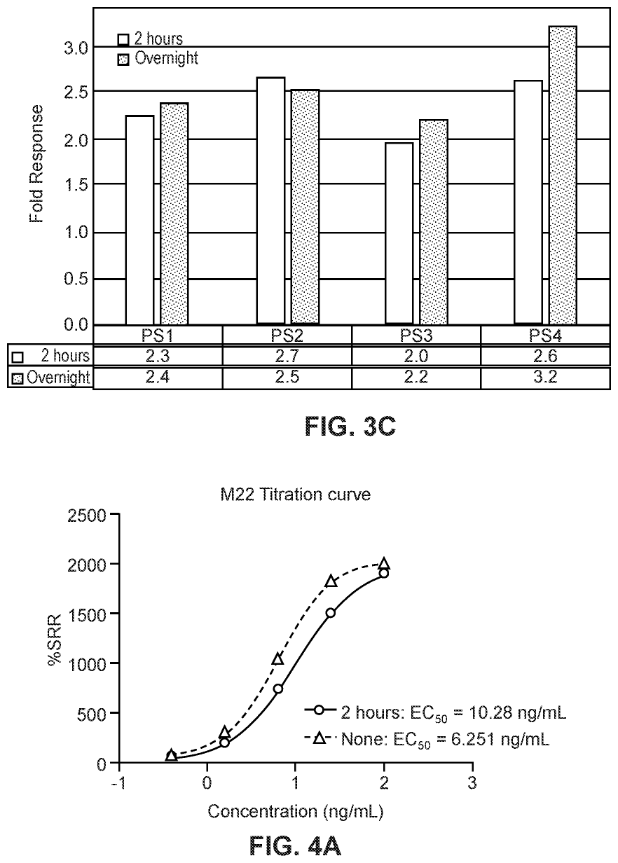

FIG. 3A shows a titration curve and calculated EC50 values obtained from the M22 standards. FIG. 3B shows signal-to-reference ratios for four negative and four positive samples. FIG. 3C shows responses for each positive sample, calculated as fold response over average values of negative samples. Both assays (overnight or 2-hour cell seeding) were able to distinguish normal samples from TSI-positive samples (FIG. 3B). Moreover, the fold increases in signals for the TSI-positive samples were comparable between both assays (FIG. 3C).

Therefore, assays performed adequately even with a reduced cell seeding time of two hours.

To evaluate whether any cell seeding is needed at all, a similar set of experiments were conducted, except that some CHO-ChR4/22F cells were seeded for two hours before incubation with substrate and reaction buffer, while some CHO-ChR4/22F cells were directly suspended in substrate and reaction buffer soon after thawing.

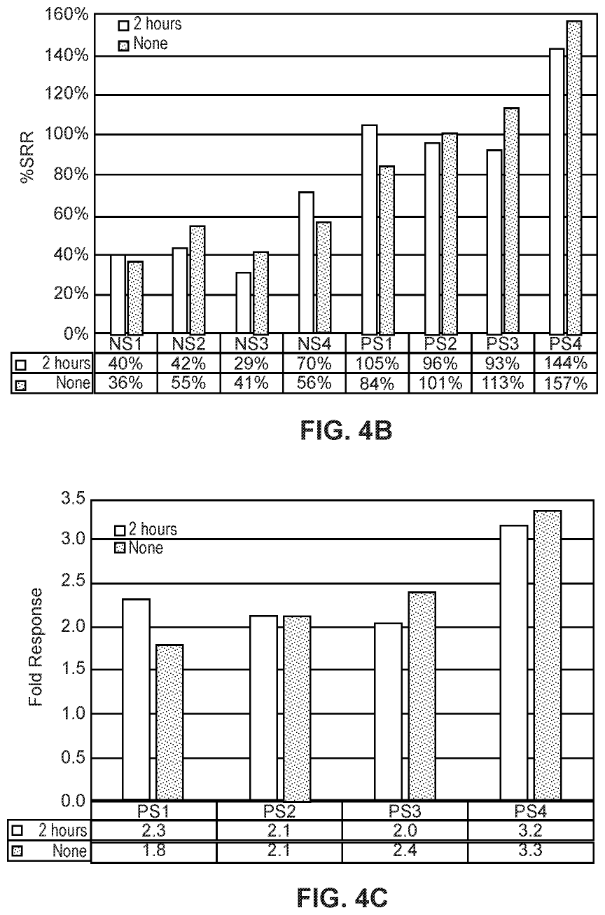

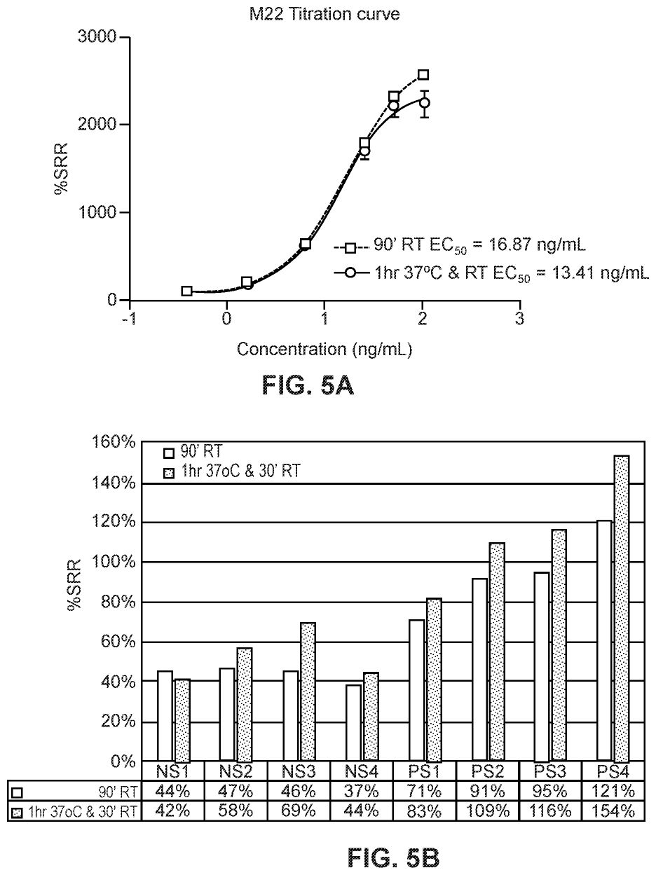

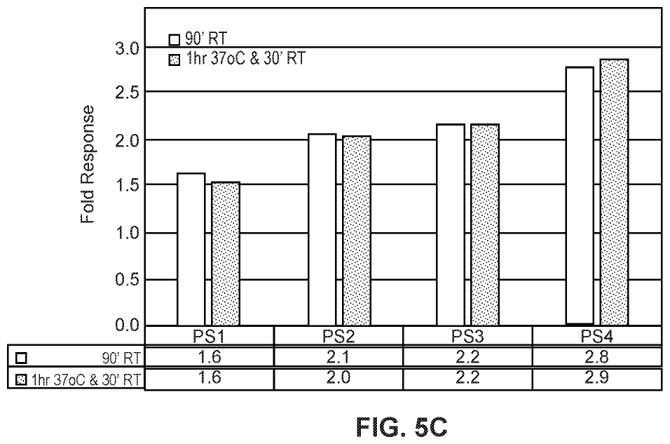

FIG. 4A shows a titration curve and calculated EC50 values obtained from the M22 standards. FIG. 4B shows signal-to-reference ratios for four negative and four positive samples. FIG. 4C shows responses for each positive sample, calculated as fold response over average values of negative samples. Both assays performed comparably with respect to ability to distinguish normal from TSI-positive samples (FIG. 5B) and with respect to fold-changes over values from normal samples (FIG. 5C).

These results suggest that the presently disclosed methods do not require a cell seeding step.

### E. Incubation Conditions

To optimize incubation conditions, a set of experiments were performed comparing two conditions for incubating CHO-ChR4/22F cells with substrate and reaction buffer. Directly after thawing, CHO-ChR4/22F cells were resuspended in 6% GLOSENSOR™ in reaction buffer and added to microwells. Ten microliters of undiluted normal or TSI-positive samples were added to each microwell, and the resulting mixture was incubated at either (1) 37° C. for one hour, followed by 30 minutes at room temperature or (2) room temperature for 90 minutes. Luminometer readings were read at the end of the incubation period. As with the experiments described in parts C and D of Example 1, M22 standards were also assayed to establish a titration curve.

FIG. 5A shows a titration curve and calculated EC50 values obtained from the M22 standards. FIG. 5B shows signal-to-reference ratios for four negative and four positive samples. FIG. 5C shows responses for each positive sample, calculated as fold response over average values of negative samples. Under both incubation conditions, the assays were able to distinguish normal from TSI-positive samples (FIG. 5B), and fold-changes over values from normal samples were comparable between the two assay conditions (FIG. 5C).

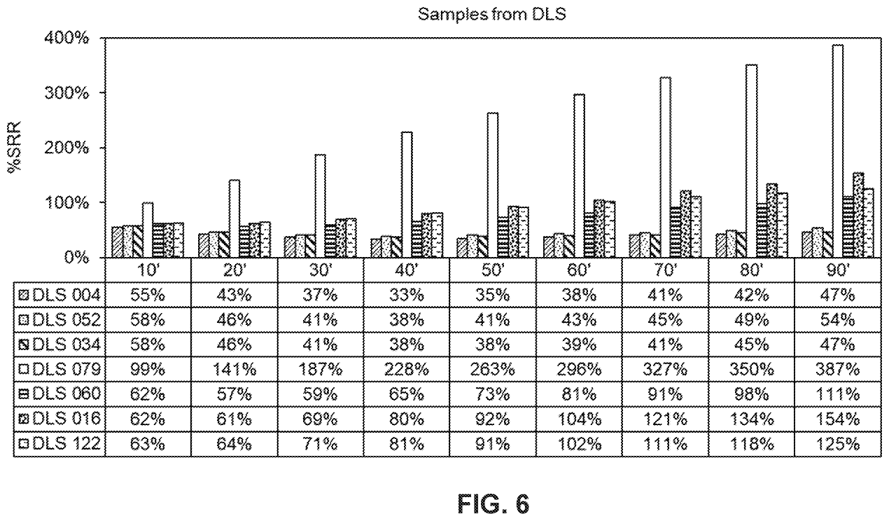

To evaluate whether a shortened incubation period would impact assay performance, a series of experiments were with various incubation times at room temperature (10 minutes, 20 minutes, 30 minutes, 40 minutes, 50 minutes, 60 minutes, 70 minutes, 80 minutes, or 90 minutes). FIG. 6 shows results from these experiments. The first three bars for each time point (samples DLS 004, DLS 052, and DLS 034) correspond to signals from normal samples; the last four bars for each time point (samples DLS 079, DLS 060, DLS 016, and DLS 122) correspond to signals from TSI-positive samples. As FIG. 6 shows, signals from TSI-positive samples can be distinguished from signals from TSI-negative samples with as little as 30 minutes of incubation time at room temperature. As expected, the separation between normal and TSI-positive samples generally increased with increasing incubation times over the time points tested.

### F. Development of a Rapid TSI Assay

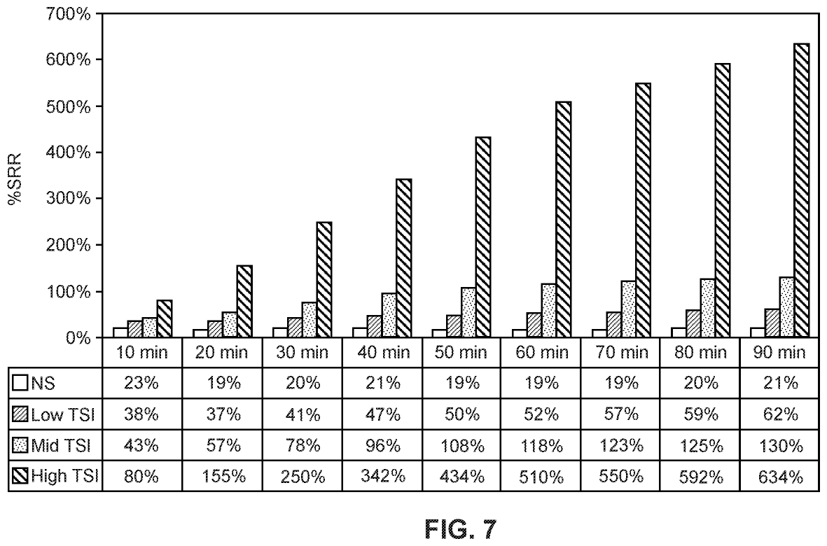

Optimum cell density, sample volume, assay temperature, and incubation time were determined using both M22 and TSI-positive serum samples. An assay reference control was prepared using bTSH at 2 mIU/mL, and assay results were reported as signal over reference ratio (% SRR). The assay was performed in a homogeneous format with only three major steps. First, 10 [symbol] l of each sample is added to duplicate wells of a white 96-well plate. Then, one vial of CHO-ChR4/22F cells is thawed at 37° C. and transferred to 10 mL of reaction buffer containing 6% luciferase substrate. Finally, 100 μl of the cell suspension (6.5×10⁵ cell/ml) is dispensed into each well. The plate is kept at room temperature and luminescence is measured every 10 minutes up to 90 minutes. The data in FIG. 7 shows that % SRR values for TSAb positive samples increase time while the % SRR value of the negative sample remains steady.

### G. Summary

The presently disclosed method obviates several steps characteristic of other TSI or TBI-detection assays and detects TSIs and/or TBIs with a short turn-around time and convenient protocol. The total turn-around time of the presently disclosed method to about or under 60 minutes, a significant decrease compared to the 21-22 hour turn-around time characteristic of other TSI or TBI-detection assays.

### Example 2

### Specificity of Assays to Detect Thyroid-Stimulating Antibodies

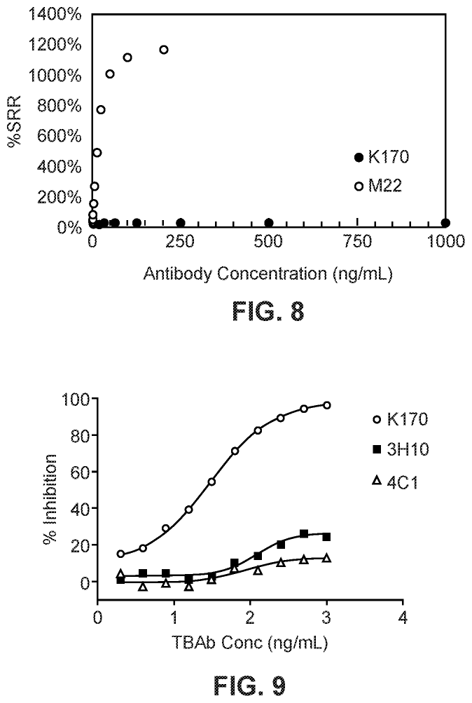

To assess the specificity of the presently disclosed TSI assay described in Example 1, serial dilutions of a thyroid-blocking antibody (K170) and of a thyroid-stimulating antibody (M22) were tested concurrently. As shown in FIG. 8, the presently disclosed TSI assay was very sensitive to M22 stimulation and reached a plateau at 50 ng/mL. In contrast, K170 did not induce luciferase activity in the presently disclosed TSI assay, even at the highest tested concentration of 1000 ng/mL (FIG. 8).

The ChR4 receptor has been shown to interact with both the stimulating antibody M22 and the blocking antibody K170 in the THYRETAIN® TSI assay. To determine whether thyroid blocking antibodies interact with the ChR4 receptor in the presently disclosed TSI assay, 10 ng/mL M22 was mixed with serially diluted (1000 ng/mL-2 ng/mL) K170 and two other mouse TSHR blocking antibodies 3H10 (DSMZ, Braunschweig, Germany) and 4C1 (Santa Cruz BioTech, Dallas, Taxes). All three blocking antibodies had an inhibitory effect on M22-induced activity of the ChR4 receptor, but the blocking activity of K170 was significantly more potent compared to the other two mouse blocking antibodies (FIG. 9).

These results support the conclusion drawn by Furmaniak et al. (Auto Immun Highlights, 2013, 4(1):11-26) and Nunez et al. (J Mol. Endocrinol. 2012, 49(2):137-151) that the TSHR antibodies with different functional activities have overlapping binding sites on the concave surface of TSHR. Because a TSHR blocking antibody does not cause a change in the intracellular concentration of cAMP, the presently disclosed TSI assay can only detect TSHR inhibitory antibody, or a net inhibitory effect of TBIs, if both types of the antibodies coexist in the samples.

However, these results demonstrate that the presently disclosed assay can be used as, or be developed into, a rapid homogenous TBI assay.

### Example 3

### An Assay for Detecting Thyroid-Blocking Autoantibodies

The present Example demonstrates a method for detecting thyroid-blocking immunoglobulins (TBIs) in a sample.

Samples and three assay controls (reference, positive and negative) are added to a multi-well microtiter plate in duplicate (10 μL per well). Reaction buffer (RB) containing an optimal concentration of bovine TSH is added to each well of the plate (50 μL per well).

CHO-ChR4/22F cells generated as described in Example 1 are thawed and resuspended in RB containing 12% GLOSENSOR™ substrate. Fifty microliters of the cell suspension is added to each well of the plate; the final concentration of the GLOSENSOR™ substrate is 6%.

Luminometer readings are taken from each well as described in Example 1, except for the following modifications.

Bovine TSH (thyroid-stimulating hormone) is added to each well (except for one or more control wells) after adding samples. The following controls are also used:
(1) “Positive control”: At least one well that does not include a sample, but does include a TSHR blocking antibody (e.g., 3H10 or K170), CHO cells, GLOSENSOR™ substrate, and reaction buffer.
(2) “Negative control”: At least one well that does not include the TSHR blocking antibody or any sample, but does include CHO cells, GLOSENSOR™ substrate, normal serum, and reaction buffer.
(3) “Reference control”: At least one well that does not include the TSHR blocking antibody or any sample, but does include bovine TSH, CHO cells, GLOSENSOR™ substrate, and reaction buffer.

Signals from wells containing samples are compared to signals from the reference control wells using Formula 1 below. A decreased signal relative to the negative control indicates presence of TBIs in a sample.

$$\% Inhibition = 1 - \frac{Samp1e RLU}{Reference RLU} \times 100 \%$$

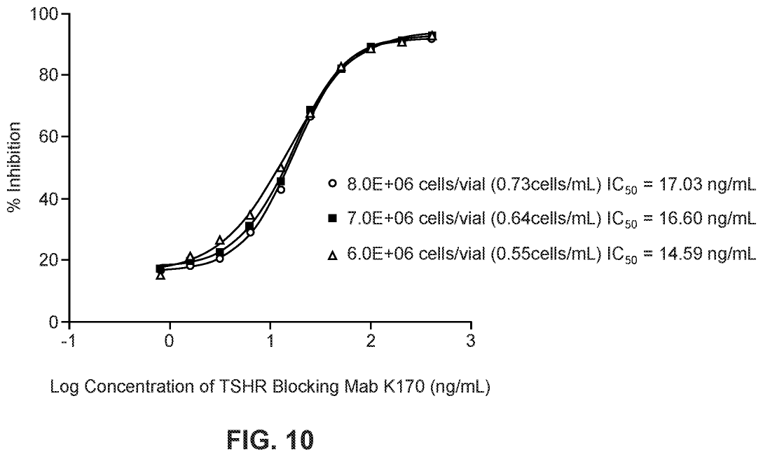

FIG. 10 shows a titration curve and calculated IC₅₀ values obtained from assays on samples containing the K170 TSHR blocking antibody. Each curve represents a titration curve on a different density of CHO-ChR4/22F transgenic cells.

### Example 4

### Reproducibility and Sensitivity of Assays

To determine the reproducibility and sensitivity of presently disclosed methods, assays were repeated on replicates of the following samples 1) reaction buffer only (blank), 2) bovine TSH in normal human serum at 2 mIU/mL (“reference sample”); 3) a normal (TSI-negative) sample; 4) a TSI-positive sample; and 5) a sample containing 3 ng/mL M22 in normal serum.

Assays were performed as described in Part E of Example 1 (hereinafter “modified TSI assay”), with incubation periods ranging from 10 minutes to 90 minutes, all at room temperature.

Table 3 presents a summary of results, including % CV. As shown in Table 3, the % CV for both RLU and % SRR were 8.1% or less for each sample.

TABLE 3
Summary of results from replicates

| | # of | Average Results | | CV % | |
|---|---|---|---|---|---|
| Serum sample | Replicate | RLU | % SRR | for RLU | for % SRR |
| Reaction | 8 | 31394 | NA | 4.1% | |
| Buffer | | | | | |
| (Blank) | | | | | |
| Reference | 8 | 149363 | NA | 3.1% | |
| Control | | | | | |
| Normal Serum | 8 | 49366 | 15% | 2.9% | 8.1% |
| (NS) | | | | | |
| TSI Positive | 8 | 244288 | 180% | 4.9% | 5.7% |
| Serum | | | | | |
| 3 ng/mL M22 | 64 | 263874 | 199% | 4.5% | 3.9% |
| in NS | | | | | |

To assess the sensitivity of the presently disclosed assay, M22 standards were evaluated using 1) the assay performed as described in Part E of Example 1, with an incubation period of 60 min at room temperature; and 2) using the THYRETAIN® TSI assay according to the manufacturer's protocol.

An amount of 100 ng/mL of M22 antibody was serially diluted into 7 concentrations and tested concurrently in both assays.

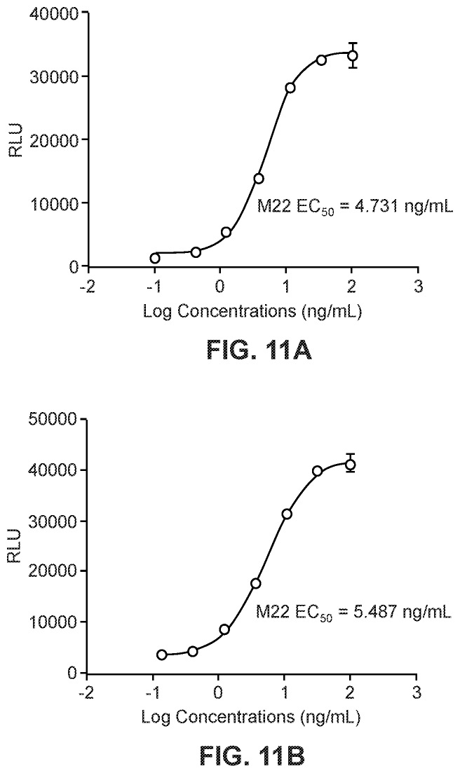

FIGS. 11A-11B show titration curves and calculated EC₅₀ values for the presently disclosed assay and for the THYRETAIN® TSI assay, respectively. The signals obtained from M22 antibody standards using the presently disclosed methods were about ten times higher than those obtained using the THYRETAIN® TSI assay, but the dose response curves from the two assays were very similar. The EC₅₀ value was 4.7 ng/mL for the modified TSI assay and about 5.5 ng/mL for the THYRETAIN® TSI assay, indicating that the analytical sensitivities of the two assays were similar.

These results indicate that the presently disclosed methods are reproducible and as sensitive as the THYRETAIN® TSI assay.

### Example 5

### TSI Detection in Clinical Samples

To assess performance of presently disclosed methods on clinical samples and to optimize assay conditions, assays were performed on clinical serum samples as described in Part E of Example 1, except for slight modifications, as discussed below. For comparison, each sample was also tested using a THYRETAIN® TSI Reporter Bioassay Kit (Quidel, Athens, Ohio) (the “THYRETAIN® TSI assay”).

### Samples

Experiments described in the present Example used 9 “normal” (negative for TSI) serum samples, 9 “low TSI” serum samples, 5 “moderate TSI” serum samples, and 5 “high TSI” serum samples from human patients, as determined by the THYRETAIN® TSI assay. (See Table 1.)

### Incubation Conditions

In one set of experiments, CHO-ChR4/22F transgenic cells were incubated with samples (and GLOSENSOR™ substrate and reaction buffer) (1) at room temperature for 90 minutes (“RT90”) or (2) at 37° C. for one hour and then room temperature for 30 minutes (“37° C.-60/RT30”). Other conditions were as described in Part E of Example 1.

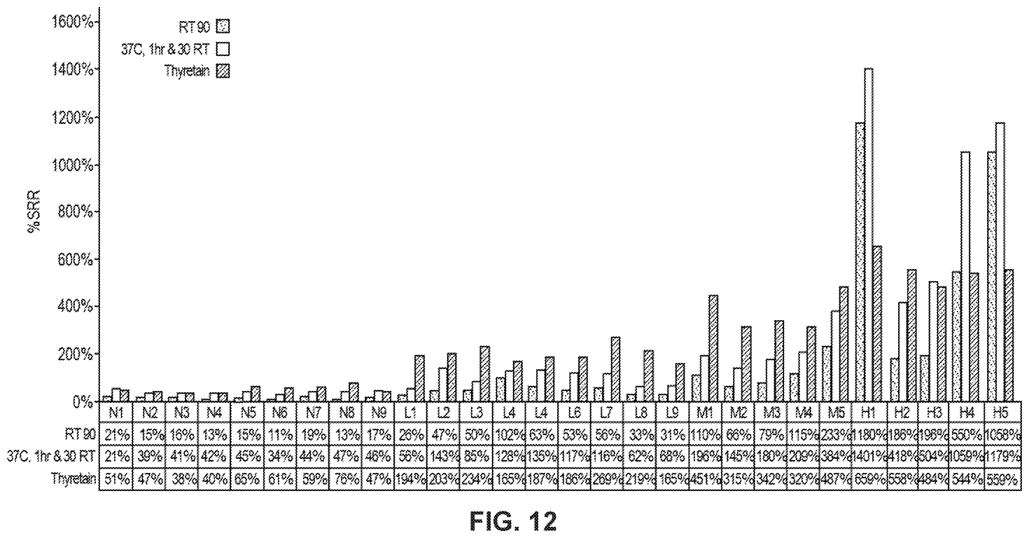

FIG. 12 shows results for both incubation conditions and for the THYRETAIN® TSI assay. As FIG. 12 shows, the separation between normal and “low TSI” samples was clearer with the RT90 assays than with the 37° C.-60/RT30 assays. In addition, both the RT90 and the 37° C.-60/RT30 assays generally distinguished “high TSI” samples better than did the THYRETAIN® TSI assay.

### Pre-Equilibration

In another set of experiments, assays were performed on clinical serum samples using (1) CHO-ChR4/22F cells (as described in Example 1) and (2) CHO-ChR4/22F cells that had been pre-equilibrated with 6% GLOSENSOR™ substrate for two hours, then frozen and subsequently thawed for use in the assay. In this set of experiments, cells were incubated for 90 minutes at room temperature.

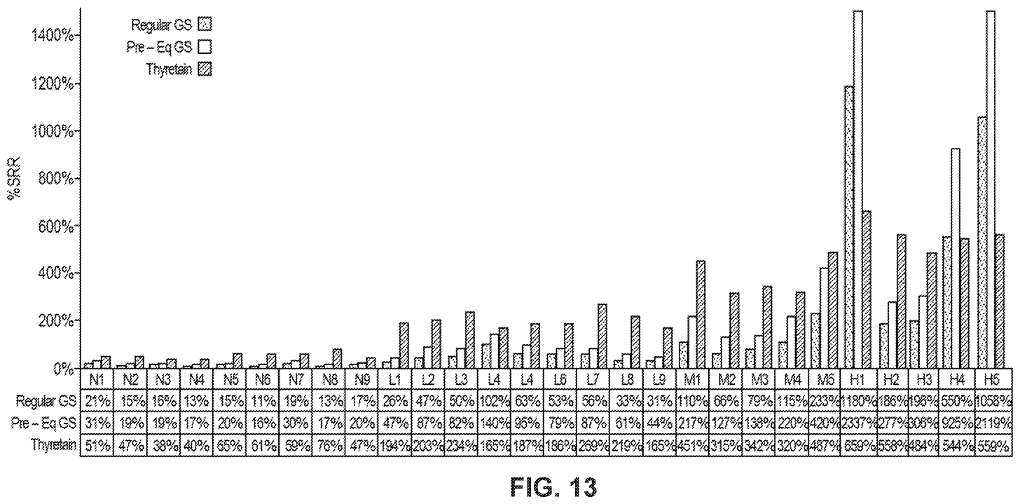

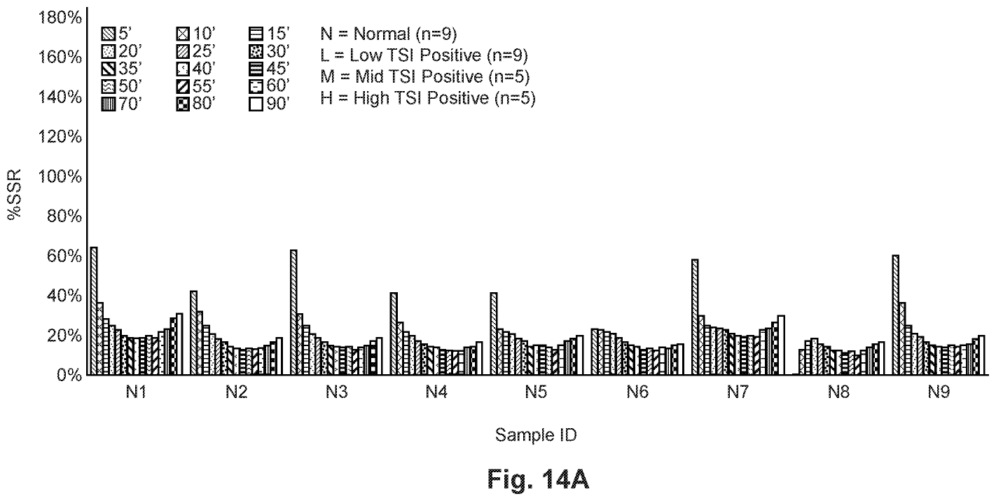

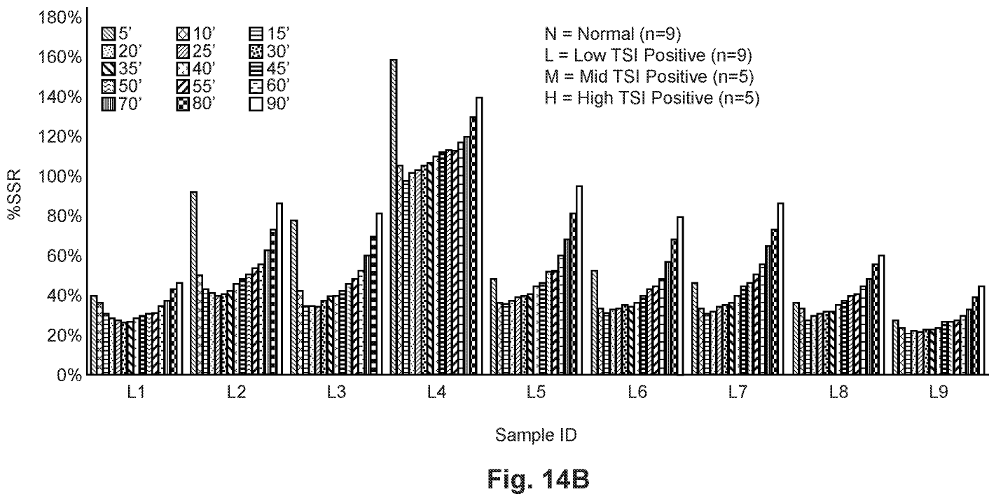

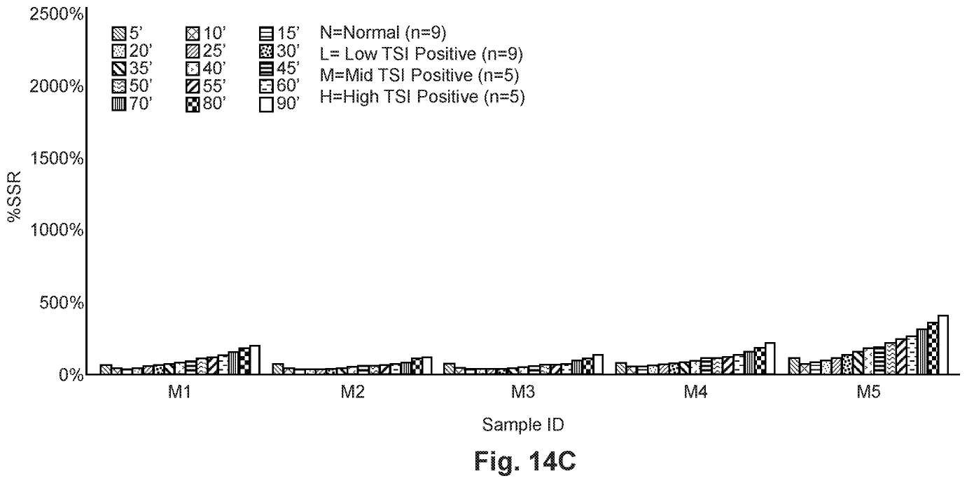

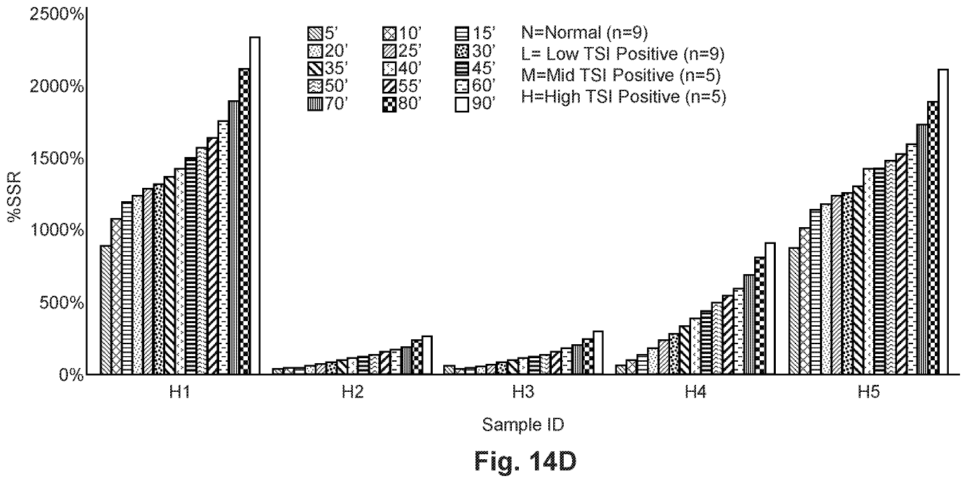

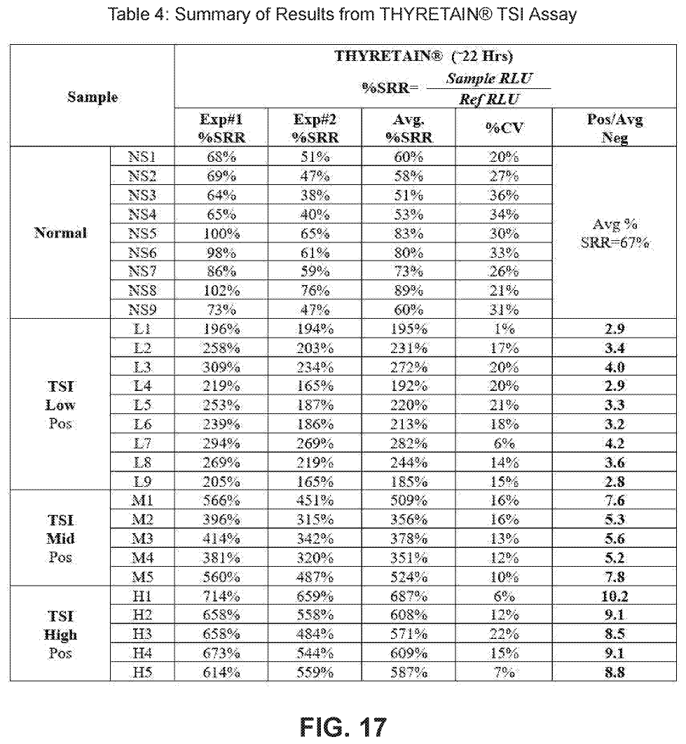

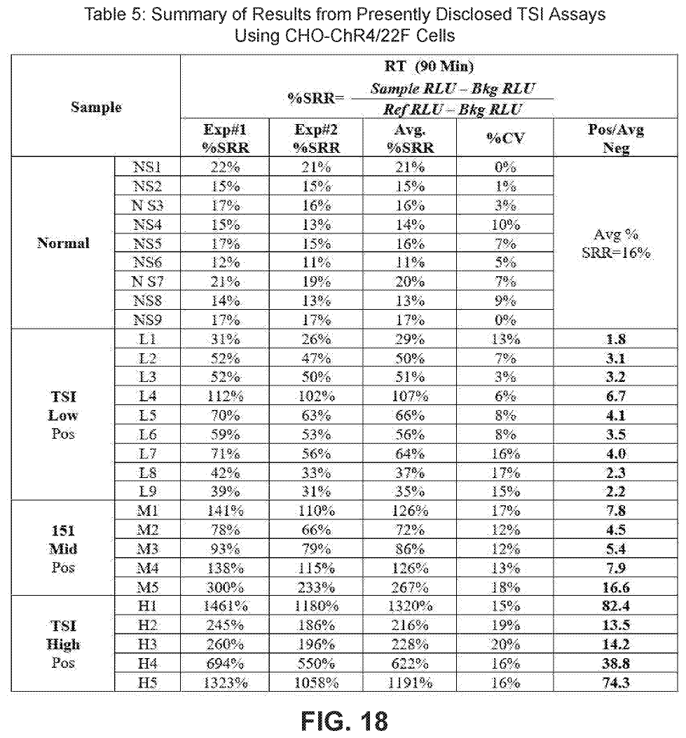

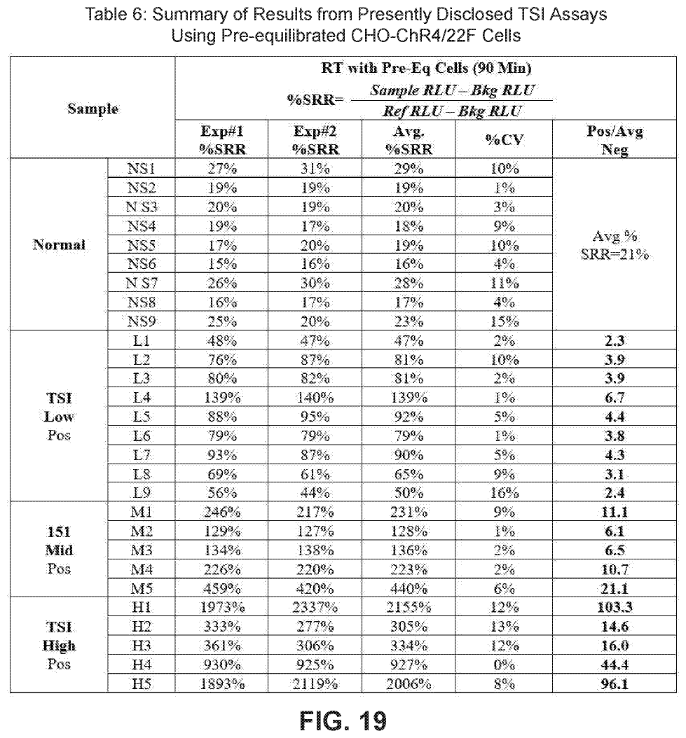

FIG. 13 and FIGS. 14A-14D show measurements from assays as described in Example 1 using CHO-ChR4/22F cells or pre-equilibrated CHO-ChR4/22F cells. For comparison, FIGS. 13 and 14A-D also show results from the THYRETAIN® TSI assay. FIG. 10 shows endpoint measurements, and FIGS. 14A-14D shows kinetic measurements taken at various time points between 5 and 90 minutes of incubation time. Tables 4-6 (FIGS. 17-19) show summary data for signal-to-response ratios obtained using the THYRETAIN®TSI Reporter BioAssay kit (Table 4; FIG. 17) and for presently disclosed assays using CHO-ChR4/22F cells and pre-equilibrated CHO-ChR4/22F cells; see Table 5 (FIG. 18) and Table 6 (FIG. 19), respectively.

As FIG. 13 and Table 4 (FIG. 17) and Table 5 (FIG. 18) show, pre-equilibrating cells with substrate increased the assay's sensitivity. In FIG. 14, the signals from negative samples tended to decrease over time, whereas the signals from TSI-positive samples tended to increase over time. Moreover, both assays (using CHO-ChR4/22F cells and pre-equilibrated CHO-ChR4/22F cells) performed at least as well as the THYRETAIN® TSI assay. For “high TSI” samples, presently disclosed assays yielded greater signals than did the THYRETAIN® TSI assay; see FIG. 13 and compare Table 4 (FIG. 17) and Table 5 (FIG. 18) to Table 3.

### Conclusion

Thus, methods of the present disclosure are able to distinguish normal, low TSI, moderate TSI, and high TSI clinical samples with results qualitatively similar to, or better than, those of the THYRETAIN® TSI assay. These results also demonstrate that (1) the present methods can distinguish normal from TSI-positive clinical samples even when cells are incubated at room temperature; and (2) pre-equilibrating cells with substrates led to enhanced assay sensitivity.

### Example 6

### Correlation Between Results from Presently Disclosed TSI Assays and Results from THYRETAIN® TSI Assays on Clinical Samples

To further evaluate the presently disclosed TSI assay's performance on clinical samples, a tentative cutoff value for the presently disclosed TSI assay was determined by measuring 145 human serum samples that had tested negative in the THYRETAIN® TSI assay. This tentative assay cutoff for the presently disclosed TSI was calculated to be 31% SRR (average % SRR value of 145 normal serum samples+2× standard deviation), and this value was used as the cutoff to identify TSI-positive samples.

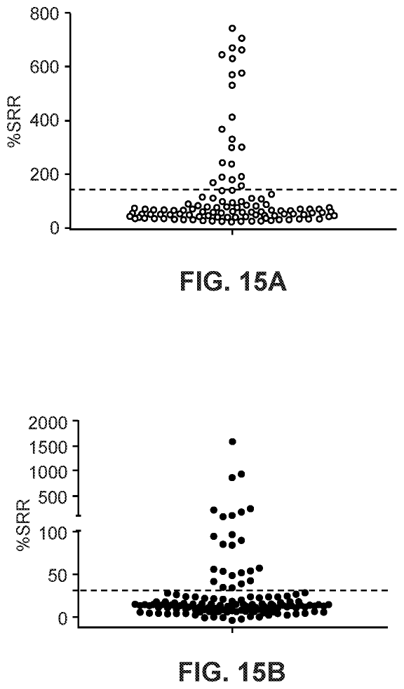

To evaluate the performance of the presently disclosed assay on samples from unselected patients with autoimmune thyroid disease (AITD), one hundred and thirty human serum samples positive for thyroid peroxidase (TPO) antibody were tested by both the presently disclosed TSI and THYRETAIN® TSI assays. Samples with a % SRR value greater than 31% were identified as positive for TSI in the presently disclosed TSI assay. According to the THYRETAIN® TSI assay instructions, samples with a % SRR value greater than 140% were identified as positive for TSI. Results from the two assays were comparable for these 130 samples, as evidenced by the fact that positive and negative percent agreement (PPA and NPA) between the two bioassays were 96% (95% Cl: 0.79-0.99) and 95% (95% Cl: 0.90-0.98) respectively (Table 7 and FIGS. 15A and 15B). Results from the two assays showed strong correlation, with a correlation coefficient R value of 0.71, despite the fact that the % SRR values generated by the presently disclosed TSI assay are much higher than those generated by the THYRETAIN® assay for the same high TSI-positive samples (data not shown).

TABLE 7
Comparison of presently disclosed TSI assay and THYRETAIN ® TSI
assay performance on 130 anti-TPO Positive Samples

| | THYRETAIN ® (+) | THYRETAIN ® (−) |
|---|---|---|
| Presently disclosed TSI | 22 | 5 |
| assay (+) | | |
| Presently disclosed TSI | 1 | 102 |
| assay (−) | | |

### Example 7

### Quantitative Detection of Thyroid-Stimulating Antibodies by the Presently Disclosed TSI Assay

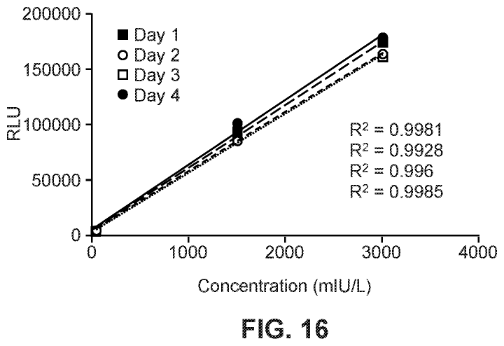

To determine whether the presently disclosed TSI assay can be used to detect TSIs quantitatively, a 3-point standard panel was developed using three concentrations of WHO International Standard for TSI. A standard panel was prepared using normal serum as a matrix and tested along with TSI-positive serum samples for 4 days. The TSI concentration of each TSI positive sample was calculated based on an equation derived from the standard curve generated in the same plate, and % CV was determined for the data obtained from four experiments performed on four different days. All four standard curves generated in four days were reproducible, with R-squared values greater than 0.99 (FIG. 16). The calculated TSI concentrations for low, moderate and high TSI-positive samples were also consistent with % CV values less than 10% (Table 8). These data indicate that the presently disclosed TSI assay has potential to be used for the quantitative measurement of TSIs in patient samples.

TABLE 8
Quantitative Detection of TSI by the presently disclosed TSI assay

| | Quantitative Result | | | | | | | | | | |
|---|---|---|---|---|---|---|---|---|---|---|---|
| Sample ID | Day 1 | | Day 2 | | Day 3 | | Day 4 | | Average | | % CV |
| Low TSI | 118 | mlU/L | 97 | mlU/L | 117 | mlU/L | 117 | mlU/L | 112 | mlU/L | 9% |
| Positive | | | | | | | | | | | |
| Moderate TSI | 709 | mlU/L | 711 | mlU/L | 713 | mlU/L | 673 | mlU/L | 701 | mlU/L | 3% |
| Positive | | | | | | | | | | | |
| High TSI | 1561 | mlU/L | 1521 | mlU/L | 1566 | mlU/L | 1527 | mlU/L | 1544 | mlU/L | 1% |
| Positive | | | | | | | | | | | |

What is claimed is:

1. A kit, comprising:
(a) transgenic cells stably transfected with:
(i) a first expression vector comprising a nucleotide sequence that encodes a chimeric thyroid stimulating hormone (TSH) receptor, and
(ii) a second expression vector comprising a synthetic nucleotide sequence that encodes a reporter, wherein the synthetic nucleotide sequence:
(1) is operably linked to a cAMP-inducible promoter; and/or
(2) further encodes a heterologous cAMP-binding protein, wherein the cAMP-binding protein is fused to the reporter; and
(b) a reaction buffer with no cell lysing agent, the buffer optionally including a substrate for the reporter,
wherein expression of the reporter is associated with an intracellular signal.

2. The kit of claim 1, wherein presence of cAMP increases expression of the reporter, thereby enhancing the signal.

3. The kit of claim 1, wherein binding of cAMP to the heterologous binding site induces a conformational change in the reporter that enhances the signal.

4. The kit of claim 1, wherein the chimeric TSH receptor comprises a human TSH receptor sequence.

5. The kit of claim 4, wherein the chimeric TSH receptor is ChR4.

6. The kit of claim 1, wherein the reporter comprises a luciferase polypeptide.

7. The kit of claim 6, wherein the luciferase polypeptide is a modified luciferase.

8. The kit of claim 7, wherein the modified luciferase is a circularly permutated luciferase.

9. The kit of claim 6, wherein the substrate comprises a luciferin.

10. The kit of claim 1, wherein the intracellular signal comprises luminescence.

11. The kit of claim 1, where in the transgenic cells comprise mammalian cells.

12. The kit of claim 11, wherein the mammalian cells comprise Chinese hamster ovary (CHO) cells or human RD cells.

13. The kit of claim 1, wherein the transgenic cells further comprise a substrate for the reporter.

14. The kit of claim 1, wherein the reaction buffer comprises polyethylene glycol and sucrose.

15. The kit of claim 14, wherein the reaction buffer further comprises albumin.

16. The kit of claim 15, wherein the albumin comprises bovine serum albumin.

17. The kit of claim 1, wherein the kit excludes a cell lysing agent.

18. A method for detecting thyroid stimulating and/or thyroid blocking autoantibodies in a sample, comprising:
(a) contacting transgenic cells with a sample suspected of comprising thyroid stimulating and/or thyroid blocking autoantibodies, wherein the transgenic cells are stably transfected with:
(i) a first expression vector comprising a nucleotide sequence that encodes a chimeric thyroid-stimulating hormone (TSH) receptor and
(ii) a second expression vector comprising a synthetic nucleotide sequence that encodes a reporter, wherein the synthetic nucleotide sequence:
(1) is operably linked to a cAMP-inducible promoter inducible, and/or
(2) further encodes a heterologous cAMP-binding protein, wherein the cAMP-binding protein is fused to the reporter; and
(b) detecting an intracellular signal associated with expression of the reporter.

19. The method of claim 18, wherein the transgenic cells further comprise a substrate for the reporter.

20. The method of claim 18, wherein the step of contacting comprises contacting in a buffer that comprises a substrate for the reporter.

21. The method of claim 20, wherein the buffer excludes a cell lysing agent.

22. The method of claim 20, further comprising a step of exposing the transgenic cells to the substrate for the reporter before the step of contacting.

23. The method of claim 18, wherein the step of contacting comprises contacting with an undiluted sample.

24. The method of claim 18, wherein step of detecting is performed less than 240 minutes, less than 180 minutes, less than 90 minutes, less than 60 minutes or less than 30 minutes after said contacting.

25. The method of claim 18 wherein the intracellular signal is detected from a composition comprising the transgenic cells and the sample.

26. The method of claim 25, wherein at least 75% of the transgenic cells in the composition are intact at the time of detecting.

27. The method of claim 18, wherein the step of contacting is performed at room temperature.

28. The method claim 18, further comprising thawing the transgenic cells before the step of contacting.

29. The method of claim 28, wherein the step of contacting is performed less than 120 minutes, less than 90 minutes, less than 60 minutes, less than 30 minutes or less than 5 minutes after said thawing.

30. The method of claim 18, wherein the step of contacting further comprises contacting the transgenic cells with a control thyroid-stimulating agent.

31. The method of claim 30, wherein the transgenic cells are contacted with the control thyroid-stimulating agent before the transgenic cells are contacted with the sample.
